SEQUENCE LISTING (34 sequences)

SEQ ID NO: 1 - protein, 58 aa, Artificial
  Amino acid sequence of human wild type HK1-18
YPVRCLLPSAHGSCADWAARWYFVASVGQCNRFWYGGCHGNANNFASEQECMSSCQGS

SEQ ID NO: 2 - protein, 51 aa, Artificial
  Synthetic
CXXXXXXXXCXXXXXXXXXXXXXXXCXXFXXXGCXXXXNXXXXXXXCXXXC

SEQ ID NO: 3 - DNA, 174 bp, Homo sapiens
taccccgtgcggtgcctgctgcccagtgcccatggctcttgcgcagactgggctgcccgctggtacttcgttgcctctgtgggccaatgtaaccgcttctggtatggcggctgccatggcaatgccaataactttgcctcggagcaagagtgcatgagcagctgccagggatct

SEQ ID NO: 4 - protein, 58 aa, Homo sapiens
YPVRCLLPPATGPCKARIIRWYFVASVGQCNRFVYGGCRGNANNFASEQECMSSCQGS

SEQ ID NO: 5 - protein, 58 aa, Homo sapiens
YPVRCLLPPATGPCRARIIRWYFVASVGQCNRFVYGGCRGNANNFASEQECMSSCQGS

SEQ ID NO: 6 - protein, 58 aa, Homo sapiens
YPVRCLLPPATGSCKAWAARWYFVASVGQCNRFWYGGCHGNANNFASEQECMSSCQGS

SEQ ID NO: 7 - protein, 58 aa, Homo sapiens
YPVRCLLPPATGPCKARAARWYFVASVGQCNRFVYGGCHGNANNFASEQECMSSCQGS

SEQ ID NO: 8 - DNA, 18 bp, Homo sapiens
acgccaagctttggagcc

SEQ ID NO: 9 - DNA, 40 bp, Homo sapiens
ccttgataggcccagccggcctaccccgtgcggtgcctgc

SEQ ID NO: 10 - DNA, 39 bp, Homo sapiens
ggatgtcaagcggccgcagatccctggcagctgctcatg

SEQ ID NO: 11 - DNA, 25 bp, Homo sapiens
ctgcagaagcaccatcaggttggtg

SEQ ID NO: 12 - DNA, 27 bp, Homo sapiens
tctcttctccaatctctcagccatggc

SEQ ID NO: 13 - DNA, 47 bp, Homo sapiens
catggctgagagattggagaagagataccccgtgcggtgcctgctgc

SEQ ID NO: 14 - DNA, 43 bp, Homo sapiens
caggctgatctagacttaagatccctggcagctgctcatgcac

SEQ ID NO: 15 - DNA, 25 bp, Homo sapiens
caggaattccattcaagaatagttc

SEQ ID NO: 16 - DNA, 30 bp, Homo sapiens
ccgtagtcatcaatttattttacataacac

SEQ ID NO: 17 - DNA, 44 bp, Homo sapiens
ctttggctaacgtcgccatggctgagagattggagaagagatac

SEQ ID NO: 18 - DNA, 42 bp, Homo sapiens
gggcagcaggcaccgcacggggtatctcttctccaatctctc

SEQ ID NO: 19 - DNA, 42 bp, Homo sapiens
ccgtgcggtgcctgctgccccctgccactggctcttgcaaag

SEQ ID NO: 20 - DNA, 44 bp, Homo sapiens
gaagtaccagcgggcagcccaggctttgcaagagccagtggcag

SEQ ID NO: 21 - DNA, 44 bp, Homo sapiens
gggctgcccgctggtacttcgttgcctctgtgggccaatgtaac

SEQ ID NO: 22 - DNA, 45 bp, Homo sapiens
catgacagccgccataccagaagcggttacattggcccacagagg

SEQ ID NO: 23 - DNA, 46 bp, Homo sapiens
ctggtatggcggctgtcatggcaatgccaataactttgcctcggag

SEQ ID NO: 24 - DNA, 40 bp, Homo sapiens
cagctgctcatgcactcttgctccgaggcaaagttattgg

SEQ ID NO: 25 - DNA, 40 bp, Homo sapiens
caagagtgcatgagcagctgccagggatcttaagtctaga

SEQ ID NO: 26 - DNA, 33 bp, Homo sapiens
cacggtcttagtttctagacttaagatccctgg

SEQ ID NO: 27 - DNA, 42 bp, Homo sapiens
ccgtgcggtgcctgctgccccctgccactggcccttgcaaag

SEQ ID NO: 28 - DNA, 44 bp, Homo sapiens
gaagtaccagcgggcagccctggctttgcaagggccagtggcag

SEQ ID NO: 29 - DNA, 45 bp, Homo sapiens
catgacagccgccatacacgaagcggttacattggcccacagagg

SEQ ID NO: 30 - DNA, 46 bp, Homo sapiens
cgtgtatggcggctgtcatggcaatgccaataactttgcctcggag

SEQ ID NO: 31 - DNA, 419 bp, Artificial
  Nucleotide sequence encoding the 212L-HKI18 fusion polypeptide
gaattccattcaagaatagttcaaacaagaagattacaaactatcaatttcatacacaatataaacgaccaaaagaatgaaggctgttttcttggttttgtccttgatcggattctgctgggcccaaccagtcactggcgatgaatcatctgttgagattccggaagagtctctgatcatcgctgaaaacaccactttggctaacgtcgccatggctgagagattggagaagagataccccgtgcggtgcctgctgcccagtgcccatggctcttgcgcagactgggctgcccgctggtacttcgttgcctctgtgggccaatgtaaccgcttctggtatggcggctgccatggcaatgccaataactttgcctcggagcaagagtgcatgagcagctgccagggatcttaagtctaga

SEQ ID NO: 32 - protein, 111 aa, Artificial
  Amino acid sequence of the 212L-HKI18 fusion polypeptide
MKAVFLVLSLIGFCWAQPVTGDESSVEIPEESLIIAENTTLANVAMAERLEKRYPVRCLLPSAHGSCADWAARWYFVASVGQCNRFWYGGCHGNANNFASEQECMSSCQGS

SEQ ID NO: 33 - protein, 111 aa, Artificial
  Amino acid sequence of the 212L-HKI18-1 fusion polypeptide
MKAVFLVLSLIGFCWAQPVTGDESSVEIPEESLIIAENTTLANVAMAERLEKRYPVRCLLPPATGSCKAWAARWYFVASVGQCNRFWYGGCHGNANNFASEQECMSSCQGS

SEQ ID NO: 34 - protein, 111 aa, Artificial
  Amino acid sequence of the 212L-HKI18-2 fusion polypeptide
MKAVFLVLSLIGFCWAQPVTGDESSVEIPEESLIIAENTTLANVAMAERLEKRYPVRCLLPPATGPCKARAARWYFVASVGQCNRFVYGGCHGNANNFASEQECMSSCQGS

(19) United States
(12) Patent Application Publication
(10) Pub. No.: US 2004/0152633 A1
(43) Pub. Date: Aug. 5, 2004

(54) Kunitz-type sequences and polypeptides

(76) Inventors: Marianne Ulrich Jorgensen, Herlev (DK); Susanne Bang, Bagsvaerd (DK); Ole Hvilsted Olsen, Bronshoj (DK); Lars Christian Petersen, Horsholm (DK)

Correspondence Address: NOVO NORDISK PHARMACEUTICALS, INC, 100 COLLEGE ROAD WEST, PRINCETON, NY, 08540, US

(21) Appl. No.: 10/721,961

(22) Filed: Nov. 25, 2003

(30) Foreign Application Priority Data
May 31, 2001 (DK) PA 2001 00859
May 31, 2002 (WO) PCT/DK02/00372

(51) Int. Cl. A61K 38/17; C07K 14/47

(52) U.S. Cl. 514/12; 530/324

(57) ABSTRACT

The invention described herein provides novel human Kunitz-type protease inhibitors; nucleic acids encoding such inhibitors; vectors and host cells comprising such nucleic acids; compositions comprising such inhibitors, cells, and/or nucleic acids; methods of producing such inhibitors, nucleic acids, vectors, compositions, and cells; and methods of inducing, promoting, and/or enhancing a physiological response in a subject by administering to the subject an amount of such an inhibitor, nucleic acid, vector, host cell, and/or composition sufficient to induce such a physiological response.

### CROSS-REFERENCE TO RELATED APPLICATIONS

This application claims the benefit priority under 35 U.S.C. 119 of Danish application no. PA 2001 00859 filed May 31, 2001 and U.S. provisional application No. 60/303,180 filed Jul. 5, 2001 and further claims priority under 35 U.S.C. 120 of international application no. PCT/DK02/00372 filed May 31, 2002, the contents of all of which are fully incorporated herein by reference.

### FIELD OF THE INVENTION

The present invention relates to novel human Kunitz-type amino acid sequences and related polypeptide protease inhibitors, nucleic acids encoding such inhibitors, and related vectors, host cells, pharmaceutical compositions, methods of production, methods of treatment, and other uses.

### BACKGROUND OF THE INVENTION

Kunitz inhibitors, are generally characterized as basic, low molecular weight proteins comprising one or more inhibitory domains (“Kunitz domains” or “Kunitz inhibitor domains”). The Kunitz domain is a folding domain of approximately 50-60 residues, which forms a central anti-parallel beta sheet and a short C-terminal helix. This characteristic domain comprises six cysteine residues that form three disulfide bonds, resulting in a double-loop structure. Between the N-terminal region and the first beta strand resides the active inhibitory binding loop. This binding loop is disulfide bonded through the P2 Cys residue to the hairpin loop formed between the last two beta strands. Isolated Kunitz domains from a variety of proteinase inhibitors have been shown to have inhibitory activity (e.g., Petersen et al., Eur. J. Biochem. 125:310-316,1996; Wagner et al., Biochem. Biophys. Res. Comm. 186:1138-1145, 1992; Dennis et al., J. Biol. Chem. 270:25411-25417, 1995).

Human proteinase inhibitors comprising one or more Kunitz domains include TFPI-1, TFPI-2, inter α trypsin inhibitor (IαI), amyloid β-protein precursor (AβPP), amyloid protein precursor homologue (APPH), placental bikunin, α3-chain of collagen type VI (CA3VI), α1-chain of collagen type VII (CA1VII), and the multi-domain protein WFIKKN.

TFPI-1, an extrinsic pathway inhibitor and a natural anticoagulant, contains three tandemly linked Kunitz inhibitor domains. The amino-terminal Kunitz domain inhibits factor VIIa, plasmin, and cathepsin G; the second domain inhibits factor Xa, trypsin, and chymotrypsin; and the third domain has no known activity. TFPI-2 has been shown to be an inhibitor of the amidolytic and proteolytic activities of human factor VIIa-tissue factor complex, factor XIa, plasma kallikrein, and plasmin (Sprecher et al., Proc. Natl. Acad. Sci. USA 91:3353-3357, 1994; Petersen et al., Biochem. 35:266-272, 1996). The ability of TFPI-2 to inhibit the factor VIIa-tissue factor complex and its relatively high levels of transcription in umbilical vein endothelial cells, placenta and liver suggests a specialized role for this protein in hemostasis. Placental bikunin is a serine proteinase inhibitor containing two Kunitz domains (Delaria et al., J. Biol. Chem. 272:12209-12214, 1997). Individual Kunitz domains of bikunin have been expressed and shown to be potent inhibitors of trypsin, chymotrypsin, plasmin, factor XIa, and tissue and plasma kallikrein (Delaria et al., ibid.). AβPP, APPH, CA3VI, CA1VII and WFIKKN contain 1 Kunitz domain as part of the protein structure.

Aprotinin (bovine pancreatic trypsin inhibitor) is a Kunitz-type inhibitor, known to inhibit various serine proteases, including trypsin, chymotrypsin, plasmin and kallikrein, and is used therapeutically in the treatment of acute pancreatitis, various states of shock syndrome, hyperfibrinolytic hemorrhage, and myocardial infarction (ycf., for instance, J. E. Trapnell et al, Brit. J. Surg. 61, 1974, p. 177; J. McMichan et al., Circulatory shock 9, 1982, p. 107; L. M. Auer et al., Acta Neurochir. 49, 1979, p. 207; G. Sher, Am. J. Obstet. Gynecol. 129, 1977, p. 164; and B. Schneider, Artzneim.-Forsch. 26, 1976, p. 1606). Administration of aprotinin in high doses significantly reduces blood loss in connection with cardiac surgery, including cardiopulmonary bypass operations (cf., for instance, B. P. Bidstrup et al., J. Thorac. Cardiovasc. Surg. 97, 1989, pp. 364-372; W. van Oeveren et al., Ann. Thorac. Surg. 44, 1987, pp. 640-645). It has previously been demonstrated (cf. H. R. Wenzel and H. Tschesche, Angew. Chem. Internat. Ed. 20, 1981, p. 295) that certain aprotinin analogues, e.g., aprotinin(1-58, Val15), exhibit a relatively high selectivity for granulocyte elastase and an inhibitory effect on collagenase. Aprotinin (1-58, Ala15) has a weak effect on elastase, while aprotinin (3-58, Arg15, Ala17, Ser42) exhibits an excellent plasma kallikrein inhibitory effect (cf. WO 89/10374).

However, when administered in vivo, aprotinin has been found to have a nephrotoxic effect in rats, rabbits, and dogs after repeated injections of relatively high doses of aprotinin (Bayer, Trasylol, Inhibitor of proteinase; E. Glaser et al. in “Verhandlungen der Deutschen Gesellschaft für Innere Medizin, 78. Kongress”, Bergmann, Munchen, 1972, pp. 1612-1614). The nephrotoxicity observed for aprotinin (which, inter alia, appears in the form of lesions) might be ascribed to the accumulation of aprotinin in the proximal tubulus cells of the kidneys as a result of the high positive net charge of aprotinin which causes it to be bound to the negatively charged surfaces of the tubuli. This nephrotoxicity makes aprotinin less suitable for clinical purposes, in particular those requiring administration of large doses of the inhibitor (such as cardiopulmonary bypass operations). Besides, aprotinin is a bovine protein which may therefore contain one or more epitopes that give rise to an undesirable immune response when administered to humans.

In addition to the drawbacks associated with aprotinin, known Kunitz-type inhibitors generally lack specificity and may have low potency. The lack of specificity in such inhibitors can result in undesirable side effects, such as nephrotoxicity that occurs after repeated injections of high doses of aprotinin. Hence, there remains a need for additional and improved Kunitz-type proteinase inhibitors. The invention provides such novel Kunitz-type inhibitors that advantageously lack the drawbacks of presently known Kunitz-type inhibitors. The invention also provides nucleic acids encoding such inhibitors, vectors and host cells comprising such nucleic acids, and methods of preparing and using all of these new and useful compositions. These and other advantages of the invention, as well as additional inventive features, will be apparent from the description of the invention provided herein.

### SUMMARY OF THE INVENTION

The invention provides a Kunitz domain peptide comprising an amino acid sequence according to the formula Cys Xaa2 Xaa3 Xaa4 Xaa5 Xaa6 Xaa7 Xaa8 Xaa9 Cys Xaa11 Xaa12 Xaa13 Xaa14 Xaa15 Xaa16 Xaa17 Xaa18 Xaa19 Xaa20 Xaa21 Xaa22 Xaa23 Xaa24 Xaa25 Cys Xaa27 Xaa28 Phe Xaa30 Xaa31 Xaa32 Gly Cys Xaa35 Xaa36 Xaa37 Xaa38 Asn Xaa40 Xaa41 Xaa42 Xaa43 Xaa44 Xaa45 Xaa46 Cys Xaa48 Xaa49 Xaa50 Cys (SEQ ID NO:2). In one aspect, the amino acid sequence is a non-naturally occurring amino acid sequence having at least about 80% amino acid sequence identity to residues 5 through 55 of wild type human HKI-18 SEQ ID NO;1. In some such aspects, the amino acid sequence can have at least about 85%, at least about 90%, or more (e.g., about 95%) amino acid sequence identity to SEQ ID NO:1. In other aspects, such an amino acid sequence can be characterized by having less than about 95%, less than about 90%, or even less than about 85% amino acid sequence identity to SEQ ID NO:1 (thus, the amino acid sequence also could be characterized as one having about 80-95%, about 80-90%, or about 80-85% amino acid sequence identity to SEQ ID NO:1). Peptides of the invention typically exhibit protease inhibitor activity.

In more particular aspects of the invention, the amino acid sequence of the novel Kunitz domain peptide can be characterized by one or more (preferably many, if not all) of the following conditions (with respect to SEQ ID NO:2): Xaa2 is an Ala, Val, Leu, Ser, Thr, Asn, Lys, Glu, Gln, Arg, Phe, Tyr, or Met residue, or is absent; Xaa3 is an Ala, Val, Leu, Ser, Thr, Asp, Glu, Gln, Phe, or Met residue, or is absent; Xaa4 is a Gly, Ala, Leu, Ser, Asp, Lys, Glu, Gln, or Pro residue, or is absent; Xaa5 is Ala, Val, Leu, Glu, Ser, Asn, Lys, Glu, Tyr, Met, Pro, or is absent; Xaa6 is an Ala, Val, Leu, Ser, Asp, Asn, Lys, Glu, Arg, Tyr, or Met residue, or is absent; Xaa7 is an Ala, Val, Thr, Asp, Lys, Glu, Gln, Arg, His, Tyr, or Pro residue, or is absent; Xaa8 is a Gly or Asp residue, or is absent; Xaa9 is a Leu, Glu, Ser, Thr, Asn, Gln, Arg, or Pro residue, or is absent; Xaa11 is a Gly, Ala, Leu, Ser, Thr, Asn, Lys, Glu, Gln Arg, or Met residue, or is absent; Xaa12 is a Gly, Ala, Thr, Asp, Glu, or His residue, or is absent; Xaa13 is a Leu, Glu, Ser, Asn, Glu, Arg, Phe, Trp, Tyr, or Met residue, or is absent; Xaa14 is an Ala, Val, Leu, Glu, Thr, Glu, Phe, or Met residue, or is absent; Xaa15 is an Ala, Val, Leu, Glu, Ser, Thr, Asn, Lys, Glu, Gln or Pro residue, or is absent; Xaa16 is a Leu, Lys, Arg, or His residue, or is absent; Xaa17 is a Phe, Trp, or Tyr residue, or is absent; Xaa18 is an Ala, His, Phe, Trp, or Tyr residue, or is absent; Xaa19 is a Phe or Tyr residue, or is absent; Xaa20 is a Val, Ser, Asp, Asn, or Arg residue, or is absent; Xaa21 is a Gly, Ala, Leu, Glu, Ser, Asn, Lys, Phe, or Pro residue, or is absent; Xaa22 is a Val, Leu, Ser, Thr, Asn, Lys, Glu, Gln, Arg, Phe, or Tyr residue, or is absent; Xaa23 is an Ala, Val, Leu, Glu, Ser, Thr, Asp, Asn, Lys, Glu, Arg, or Tyr residue, or is absent; Xaa24 is a Gly, Asn, Lys, Glu, Gln, Arg, Tyr, or Met residue, or is absent; Xaa25 is an Ala, Leu, Glu, Ser, Thr, Lys, Glu, Gln, Arg, or His residue, or is absent; Xaa27 is an Ala, Val, Ser, Thr, Asp, Asn, Lys, Glu, Gln, Arg, or His residue, or is absent; Xaa28 is an Ala, Leu, Ser, Thr, Asn, Lys, Glu, Gln, Arg, Met, or Pro residue, or is absent; Xaa30 is an Ala, Val, Leu, Glu, Thr, Lys, Gln Phe, Trp, or Pro residue, or is absent; Xaa31 is a Ser, Phe, or Tyr residue, or is absent; Xaa32 is a Gly, Ser, Thr, or Arg residue, or is absent; Xaa35 is a Gly, Leu, Asp, Asn, Glu, Gln, Arg, His, Tyr, or Met residue, or is absent; Xaa36 is a Gly, Ala, or Arg residue, or is absent; Xaa37 is a Ser, Asp, Asn, or Lys residue, or is absent; Xaa38 is a Gly, Ala, Ser, Asp, Asn, Lys, Glu, Gln or Arg residue, or is absent; Xaa40 is a Ser, Asn, Lys, or Arg residue, or is absent; Xaa41 is a Phe or Tyr residue, or is absent; Xaa42 is a Gly, Ala, Val, Leu, Thr, Asp, Asn, Lys, Glu, Gln, Arg, His, Tyr, or Pro residue, or is absent; Xaa43 is a Ser, Thr, Asp, Asn, Glu, or Arg residue, or is absent; Xaa44 is an Ala, Leu, Lys, Glu, Gln, Arg, or Trp residue, or is absent; Xaa45 is an Ala, Asp, Lys, Glu, or Gln residue, or is absent; Xaa46 is an Ala, Ser, Thr, Asp, Asn, Lys, Glu, Gln or Tyr residue, or is absent; Xaa48 is a Leu, Ile, Glu, Asp, Lys, Glu, Gln, Arg, or Met residue, or is absent; Xaa49 is a Gly, Ala, Leu, Ser, Thr, Asp, Asn, Lys, Glu, Gln, or Arg residue, or is absent; Xaa50 is an Ala, Ser, Thr, Val, Glu, Lys, Arg, Phe, or Met residue, or is absent. Typically, only a few residues in the Kunitz domain sequence defined by the sequence pattern of SEQ ID NO:2 will be “absent” (e.g., about 10 residues or less, about 5 residues or less, or about 3 residues or less).

The invention further provides novel nucleic acids encoding such polypeptides, vectors and cells comprising such nucleic acids, and methods of using these compositions (alone or in combination) in the induction, promotion, and/or enhancement of one or more physiological responses associated with a Kunitz domain in a subject (e.g., in the prevention and/or treatment of disease in a human subject).

### BRIEF DESCRIPTION OF THE FIGURES

The present invention is described in further detail in the examples with reference to the appended drawings wherein:

FIG. 1 shows the 174 bp DNA sequence encoding the human wild type HKI-18 Kunitz domain.

FIG. 2 shows the 58 amino acid sequence of the human wild type HKI-18 Kunitz domain.

FIG. 3 shows plasmid pMaUJ72 where the human wild type HKI-18 open reading frame has been cloned in pCANTAB 5E (Amersham Pharmacia) as described in Example 1, “Cloning of human wild type HKI-18”. Only restriction sites relevant for the construction of the plasmid described in the text have been indicated FIG. 4 shows plasmid pMaUJ238. The plasmid contains an expression cassette comprising DNA encoding a fusion between the 212L leader and human wild type HKI-18. The plasmid was constructed as described in Example 1, “Construction of 212L-HKI18 fusion”. Only restriction sites relevant for the construction of the plasmid described in the text have been indicated.

FIG. 5 shows an example on the construction of an expression cassette comprising DNA encoding a fusion between the 212L leader and human wild type HKI-18. PCR product 1 includes the 212L leader sequence and the Lys-Arg Kex2p cleavage site. PCR product 2 contains the human wild type HKI-18 open reading frame. PCR products 1 and 2 are used in a new PCR where the overlap extension ensures the resulting gene SOE product. The 5′ end of primer b (corresponding to oMaUJ88) is complementary to the 3′ end of primer c (corresponding to oMaUJ89). In the gene SOE reaction the two independent PCR products 1 and 2 are incubated with the primers a (corresponding to oMaUJ87) and d (corresponding to oMaUJ90).

FIG. 6 shows the pEA314 yeast plasmid. The plasmid contains an expression cassette comprising an EcoRI-XbaI fragment inserted between the transcription-promoter and the transcription-terminator of the S. cerevisiae TPI gene. POT is the selective marker, the Schizosaccharomyces pombe triosephosphate isomerase gene. Only restriction sites relevant for the description of pEA314 and for the construction of the 212L-HKI18 and alpha*L-HKI18 plasmids have been indicated.

FIG. 7 shows the nucleotide sequence and corresponding amino acid sequence of the 420 bp sequence EcoRI-XbaI encoding the 212L-HKI18 fusion polypeptide. The 212L leader sequence is underlined.

FIG. 8 shows 212L-HKI18-1 (212L-HKI18 P9, T11, K15, A16) and 212L-HKI18-2 (212L-HKI18 P9, T11, P13, K15, A16, R17, V34). The amino acid substitutions are shown with underlined and highlighted letters.

### DETAILED DESCRIPTION OF THE INVENTION

As indicated in the foregoing Background and Summary, the present invention relates to novel polypeptides comprising at least one novel Kunitz domain (or Kunitz amino acid sequence). Among other things, the novel polypeptides of the invention can be used as protease inhibitors.

In one aspect, the invention provides Kunitz domain polypeptide comprises at least one Kunitz domain amino acid sequence according to (or falling within the definition of) the sequence pattern of SEQ ID NO:2. The amino acid sequence can be any sequence of amino acids falling within this pattern that provide a functional Kunitz domain (e.g., a Kunitz domain exhibiting protease inhibitor activity). Typically, the amino acid sequence is at least about 80% identical (e.g., about 85% identical, about 90% identical, about 95% identical, or more identical) to the sequence defined by residues 5 through 55 of wild type human HKI-18 (SEQ ID NO:1) (also referred to herein as “HKI-18 polypeptide”). The wild type human HKI-18 Kunitz domain has the amino acid sequence: Tyr Pro Val Arg Cys Leu Leu Pro Ser Ala His Gly Ser Cys Ala Asp Trp Ala Ala Arg Trp Tyr Phe Val Ala Ser Val Gly Gln Cys Asn Arg Phe Trp Tyr Gly Gly Cys His Gly Asn Ala Asn Asn Phe Ala Ser Glu Gln Glu Cys Met Ser Ser Cys Gln Gly Ser (SEQ ID NO:1).

The term “a polypeptide” as used herein, means a molecule comprising a polymer of amino acid residues (typically joined by peptide bonds), whether produced naturally or synthetically. A “polypeptide” may comprise one or more polypeptide chains. Unless otherwise stated or clearly otherwise contemplated by context, the terms polypeptide, protein, and peptide are intended to be used interchangeably (indeed, many of the polypeptides described herein are under 100 amino acid residues in length). A polypeptide may comprise non-peptidic components, such as linked carbohydrate groups. Carbohydrates and other non-peptidic substituents may be added to a polypeptide by the cell in which the polypeptide is produced and, accordingly, with the type of cell that produced the polypeptide. Alternatively, carbohydrates or other groups can be added by chemical synthesis techniques. Polypeptides are defined herein in terms of their amino acid backbone structures; substituents such as carbohydrate groups are generally not specified, but may be present nevertheless.

In more particular aspects, the invention provides a polypeptide comprising at least one Kunitz domain amino acid sequence that is at least about 90% identical to residues 5 through 55 of SEQ ID NO:1. In a further particular aspect, the invention provides a polypeptide that comprises at least one amino acid sequence that is at least 95% identical to residues 5 through 55 of SEQ ID NO:1.

Also or alternatively, the novel Kunitz domain sequence can be characterized by one or more (preferably many, or most, if not all) of the following conditions (with respect to SEQ ID NO:2): Xaa2 is an Ala, Val, Leu, Ser, Thr, Asn, Lys, Glu, Gln, Arg, Phe, Tyr, or Met residue, or is absent; Xaa3 is an Ala, Val, Leu, Ser, Thr, Asp, Glu, Gln Phe, or Met residue, or is absent; Xaa4 is a Gly, Ala, Leu, Ser, Asp, Lys, Glu, Gln, or Pro residue, or is absent; Xaa5 is Ala, Val, Leu, Glu, Ser, Asn, Lys, Glu, Tyr, Met, Pro, or is absent; Xaa6 is an Ala, Val, Leu, Ser, Asp, Asn, Lys, Glu, Arg, Tyr, or Met residue, or is absent; Xaa7 is an Ala, Val, Thr, Asp, Lys, Glu, Gln, Arg, His, Tyr, or Pro residue, or is absent; Xaa8 is a Gly or Asp residue, or is absent; Xaa9 is a Leu, Glu, Ser, Thr, Asn, Gln, Arg, or Pro residue, or is absent; Xaa11 is a Gly, Ala, Leu, Ser, Thr, Asn, Lys, Glu, Gln, Arg, or Met residue, or is absent; Xaa12 is a Gly, Ala, Thr, Asp, Glu, or His residue, or is absent; Xaa13 is a Leu, Glu, Ser, Asn, Glu, Arg, Phe, Trp, Tyr, or Met residue, or is absent; Xaa14 is an Ala, Val, Leu, Glu, Thr, Glu, Phe, or Met residue, or is absent; Xaa15 is an Ala, Val, Leu, Glu, Ser, Thr, Asn, Lys, Glu, Gln or Pro residue, or is absent; Xaa16 is a Leu, Lys, Arg, or His residue, or is absent; Xaa17 is a Phe, Trp, or Tyr residue, or is absent; Xaa18 is an Ala, His, Phe, Trp, or Tyr residue, or is absent; Xaa19 is a Phe or Tyr residue, or is absent; Xaa20 is a Val, Ser, Asp, Asn, or Arg residue, or is absent; Xaa21 is a Gly, Ala, Leu, Glu, Ser, Asn, Lys, Phe, or Pro residue, or is absent; Xaa22 is a Val, Leu, Ser, Thr, Asn, Lys, Glu, Gln, Arg, Phe, or Tyr residue, or is absent; Xaa23 is an Ala, Val, Leu, Glu, Ser, Thr, Asp, Asn, Lys, Glu, Arg, or Tyr residue, or is absent; Xaa24 is a Gly, Asn, Lys, Glu, Gln, Arg, Tyr, or Met residue, or is absent; Xaa25 is an Ala, Leu, Glu, Ser, Thr, Lys, Glu, Gln, Arg, or His residue, or is absent; Xaa27 is an Ala, Val, Ser, Thr, Asp, Asn, Lys, Glu, Gln, Arg, or His residue, or is absent; Xaa28 is an Ala, Leu, Ser, Thr, Asn, Lys, Glu, Gln, Arg, Met, or Pro residue, or is absent; Xaa30 is an Ala, Val, Leu, Glu, Thr, Lys, Gln Phe, Trp, or Pro residue, or is absent; Xaa31 is a Ser, Phe, or Tyr residue, or is absent; Xaa32 is a Gly, Ser, Thr, or Arg residue, or is absent; Xaa35 is a Gly, Leu, Asp, Asn, Glu, Gln, Arg, His, Tyr, or Met residue, or is absent; Xaa36 is a Gly, Ala, or Arg residue, or is absent; Xaa37 is a Ser, Asp, Asn, or Lys residue, or is absent; Xaa38 is a Gly, Ala, Ser, Asp, Asn, Lys, Glu, Gln or Arg residue, or is absent; Xaa40 is a Ser, Asn, Lys, or Arg residue, or is absent; Xaa41 is a Phe or Tyr residue, or is absent; Xaa42 is a Gly, Ala, Val, Leu, Thr, Asp, Asn, Lys, Glu, Gln, Arg, His, Tyr, or Pro residue, or is absent; Xaa43 is a Ser, Thr, Asp, Asn, Glu, or Arg residue, or is absent; Xaa44 is an Ala, Leu, Lys, Glu, Gln, Arg, or Trp residue, or is absent; Xaa45 is an Ala, Asp, Lys, Glu, or Gln residue, or is absent; Xaa46 is an Ala, Ser, Thr, Asp, Asn, Lys, Glu, Gln or Tyr residue, or is absent; Xaa48 is a Leu, Ile, Glu, Asp, Lys, Glu, Gln, Arg, or Met residue, or is absent; Xaa49 is a Gly, Ala, Leu, Ser, Thr, Asp, Asn, Lys, Glu, Gln or Arg residue, or is absent; Xaa50 is an Ala, Ser, Thr, Val, Glu, Lys, Arg, Phe, or Met residue, or is absent. Typically, the number of “absent” residues in this set will be few (e.g., about 10 or less, about 5 or less, or about 3 or less). Where an “Xaa” residue is not specified, that position can be filled by an amino acid residue (typically, a naturally occurring amino acid) or absence of a residue.

In yet more particular aspects of the invention, the Kunitz domain amino acid sequence is defined according to one or more of the following conditions: Xaa5 of SEQ ID NO:2 is Pro; Xaa7 of SEQ ID NO:2 is Thr; Xaa9 of SEQ ID NO:2 is Pro; Xaa11 of SEQ ID NO:2 is Arg or Lys; Xaa12 of SEQ ID NO:2 is Ala; Xaa13 of SEQ ID NO:2 is Arg; Xaa14 of SEQ ID NO:2 is Ile; Xaa15 of SEQ ID NO:2 is Ile; Xaa30 of SEQ ID NO:2 is Val; and Xaa35 of SEQ ID NO:2 is Arg.

The HKI-18 Kunitz domain comprises six cysteine residues that form three disulfide bonds, resulting in a double-loop structure. Referring to SEQ ID NO:1, disulfide bonds in the HKI-18 Kunitz domain are formed by paired cysteine residues Cys5-Cys55; Cys14-Cys38; and Cys30-Cys51. Referring to SEQ ID NO:2, disulfide bonds in the HKI-18 Kunitz domain are formed by paired cysteine residues Cys1-Cys51; Cys10-Cys34; and Cys26-Cys47. Non-naturally occurring Kunitz domain amino acid sequences (e.g., variants of residues 5-55 of SEQ ID NO:1) desirably retain a similar set of cysteine residues with similar spacing between the residues and similar resulting secondary structure.

The term “variant”, as used herein with reference to an amino acid sequence or polypeptide consisting or consisting essentially of such a sequence refers to a sequence wherein one or more amino acid residues have been deleted, inserted, added (N-terminal and/or C-terminal), and/or substituted in relation to the amino acid sequence it is considered to “vary” from (which may be referred to as the “parent” amino acid sequence or polypeptide). Variants preferably retain at least some of the biological activity of the parent (e.g., proteolytic inhibition activity similar to SEQ ID NO: 1) and/or retain a structure similar to the parent (e.g., a secondary structure similar to that produced by the disulfide bonds of SEQ ID NO:1).

In another aspect, the invention provides a polypeptide comprising at least one amino acid sequence corresponding to at least a portion of SEQ ID NO:1, typically ranging from residue 1 to about residue 55 of SEQ ID NO:1, or a residue at or about residue 5, to about residue 55 of SEQ ID NO:1 (e.g., amino acids 1-58 of SEQ ID NO:1). Such a polypeptide is preferably at least substantially, if not totally isolated. The term “isolated”, when applied to a polypeptide, denotes a polypeptide that is free of other extraneous or unwanted biological molecules (biomolecules). In the context of a naturally occurring polypeptide, an “isolated” polypeptide typically is free of the biological molecules that the polypeptide is normally associated with and/or is found in a condition other than its normal native environment, such as apart from blood and animal tissue. Thus, an isolated polypeptide typically is substantially free of other polypeptides, particularly other polypeptides of animal origin. In some aspects, polypeptides of the invention are provided in a highly purified form, i.e., about 90% or more pure, about 95% or more pure, or even about 99% or more pure. An isolated polypeptide can be present in any suitable physical form, such as multimeric forms (e.g., dimers) or alternatively glycosylated and/or otherwise derivatized forms.

In more particular aspects, the invention provides a polypeptide comprising SEQ ID NO:4, SEQ ID NO:5, SEQ ID NO:6, or SEQ ID NO:7. Polypeptides of the invention also can consist essentially or consist entirely of these sequences, respectively.

In another aspect, the invention provides an isolated polypeptide obtained by a method comprising providing a host cell comprising a nucleic acid (e.g., a polynucleotide construct) encoding a polypeptide comprising the amino acid sequence of SEQ ID NO:2 or a variant of SEQ ID NO:2 that is at least about 80% identical (e.g., about 85%, about 90%, about 95%, or more identical) to residues 5 through 55 of SEQ ID NO:1, cultivating the host cell in an appropriate growth medium under conditions permissive for the expression of the polypeptide, and recovering the polypeptide from the culture medium.

The nucleic acids of the present invention can be any suitable type of nucleic acid resulting in the production of the polypeptide (e.g., a nucleic acid can be single or double stranded DNA, RNA, DNA/RNA hybrids, or nucleic acid comprising one or more non-naturally occurring bases or other modifications, such as a phosphothioate backbone). Typically, the more specific term “polynucleotide” denotes a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. The length of a polynucleotide molecule typically is given herein in terms of nucleotides (abbreviated “nt”) or base pairs (abbreviated “bp”). The term “nucleotides” is used for both single- and double-stranded molecules where the context permits. When the term is applied to double-stranded molecules it is used to denote overall length and will be understood to be equivalent to the term “base pairs”. The two strands of a double-stranded polynucleotide may differ slightly in length and that the ends thereof may be staggered as a result of enzymatic cleavage; thus all nucleotides within a double-stranded polynucleotide molecule may not be paired. Such unpaired ends will usually not exceed about 20 nt in length.

The term “HKI-18 gene” as used herein, means a gene encoding a HKI-18 polypeptide.

The term “host cell”, as used herein, represents any cell, including hybrid cells, in which heterologous DNA can be expressed. Typical host cells include, but are not limited to, bacterial cells, insect cells, yeast cells, and mammalian cells (including human cells, such as BHK, CHO, HEK, and COS cells). Examples of suitable yeasts cells include cells of Saccharomyces spp. or Schizosaccharomyces spp., in particular strains of Saccharomyces cerevisiae or Saccharomyces kluyveri. A preferred strain of Saccharomyces cerevisiae is the strain MT663 (MATa/MATα pep4-3/pep4-3 HIS4/his4 tpi::LEU2/tpi::LEU2 Cir⁺). Strain MT663 is deposited in the Deutsche Sammiung von Mikroorganismen und Zellkulturen with deposit number DSM 6278 in connection with filing of WO 92/11378.

In yet another aspect, the invention provides a nucleic acid encoding a Kunitz domain polypeptide comprising an amino acid sequence according to the sequence pattern of SEQ ID NO:2 that is at least about 80% identical (e.g., about 85% identical, about 90% identical, about 95% identical, or more) to residues 5 through 55 of SEQ ID NO:1. The nucleic acid can be in the form of (e.g., as in the case of a linear expression element) or form part of a nucleic acid delivery vehicle (i.e., a “vector”), such as a DNA vector (e.g., a plasmid DNA vector, which can be a “naked DNA” vector, DNA-peptide conjugate vector, liposome-encapsulated DNA vector, etc.). A “vector” can comprise any number of additional sequences and/or components that promote host cell targeting and/or transfection (or other uptake and/or delivery) and/or that promote expression of desired sequences in a host cell. A vector can be characterized as a nucleic acid entity capable of the amplification in a host cell. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. Also or alternatively, the vector can be characterized by partial or substantially total replication deficiency in a particular host cell (e.g., the vector may be a replication deficient viral vector, such as a replication deficient adenoviral vector or adeno-associated virus vector, which requires a complementation cell for replication). The choice of vector will often depend on the host cell into which it is to be introduced. Vectors include, but are not limited to plasmid vectors, phage vectors, viruses, and cosmid vectors, numerous examples of which are known in the art. Vectors usually contain a replication origin and at least one selectable gene, i.e., a gene which encodes a product which is readily detectable or the presence of which is essential for cell growth.

In a further aspect, the invention relates to a host cell comprising a nucleic acid encoding a Kunitz domain polypeptide comprising at least one amino acid sequence according to the sequence pattern of SEQ ID NO:2 that is at least about 80% identical (e.g., about 90%, about 95%, or more identical) to residues 5 through 55 of SEQ ID NO:1. In yet another aspect, the invention provides a host cell comprising a vector comprising such a nucleic acid.

In a further aspect, the invention provides a method for producing an isolated polypeptide comprising at least one amino acid sequence according to the sequence pattern of SEQ ID NO:2, wherein the sequence is at least about 80% identical (e.g., about 85%, about 90%, about 95%, or more identical) to residues 5 through 55 of SEQ ID NO:1, the method comprising cultivating a host cell comprising a nucleic acid encoding the polypeptide in an appropriate growth medium under conditions allowing expression of the polypeptide and recovering the polypeptide from the culture medium. Techniques for transfecting cells with nucleic acids (as naked molecules—e.g., by electroporation or via a vector), culturing such cells, and isolating polypeptides therefrom (e.g., by centrifugation, Western blot, immunoprecipitation, and/or chromatographic techniques) are routinely used in the art.

In a further aspect, the invention relates to a composition comprising a polypeptide, preferably an isolated polypeptide, of the invention in combination with one or more pharmaceutically acceptable vehicles, carriers, diluents, and/or excipients. Compositions for pharmaceutical use can comprise any suitable vehicle, carrier, diluent, excipient, or combination thereof. Numerous examples of suitable vehicles, carriers, diluents, and excipients are known in the art.

In a further aspect, the invention relates to a method of promoting, enhancing, and/or inducing at least one physiological response in a subject associated with the prevention and/or treatment of systemic inflammatory response syndrome (SIRS), disseminated intravascular coagulation (DIC), acute respiratory distress syndrome (ARDS), sepsis, ischemia/reperfusion injury, acute pancreatitis, trauma, shock syndrome, hyperfibrinolytic hemorrhage, and/or myocardial infarction. In another aspect, the invention provides a method of reducing and preferably preventing blood loss in a subject, such as during major surgery.

The term “treatment”, as used herein, generally means the administration of an effective amount of a polypeptide of the invention with the purpose and effect of preventing any symptoms or disease state to develop or with the purpose of reducing, easing, alleviating, and/or curing such symptoms or disease states if already developed. The term “treatment” is thus, in its broadest sense, meant to include prophylaxis.

The term “subject” as used herein is intended to mean any animal, in particular mammals, such as humans, and may, where appropriate, be used interchangeably with the term “patient”.

In one aspect of the invention, the host cell is a eukaryotic cell. In a further aspect of the invention, the host cell is of mammalian origin. In a further aspect of the invention, the host cell is a yeast cell. In a further aspect of the invention, the host cell is a strain of Saccharomyces cerevisiae.

Polypeptides of the invention advantageously exhibit proteinase inhibiting activity. In a particular aspect, the invention provides a novel Kunitz domain polypeptide, as described above, that inhibits at least one of the proteases selected from the group consisting of chymotrypsin, elastase, cathepsin G, proteinase 3, plasmin, plasma kallikrein, glandular kallikrein, and trypsin.

The polypeptides of the invention can be any suitable length and can comprise any suitable number of Kunitz domain sequences of any suitable length (e.g., the polypeptide can comprise one, two, or more novel Kunitz domain sequences according to the sequence pattern of SEQ ID NO:2 in addition, optionally, to one or more other Kunitz domain sequences). Typically, a Kunitz domain amino acid sequence of the invention is about 50-70 or about 50-80 (e.g., 51-81 or 61-67) amino acids in length.

The level of identity between amino acid sequences can be determined using the “FASTA” similarity search algorithm of Pearson and Lipman (Proc. Natl. Acad. Sci. USA 85:2444, 1988) and Pearson (Meth. Enzymol. 183:63, 1990) or the BLAST algorithm (e.g., as incorporated into the San Diego Supercomputer Center Biotechnology Workbench and/or the National Center for Biotechnology Information's publicly accessible BLAST program).

For the preparation of recombinant HKI-18 polypeptides, a cloned human wild-type polynucleotide sequence encoding HKI-18 can be used. This sequence may be modified to encode a desired HKI-18 polypeptide. Amino acid sequence alterations may be accomplished by a variety of techniques. Modification of the DNA sequence may be by site-specific mutagenesis. Techniques for site-specific mutagenesis are well known in the art and are described by, for example, Zoller and Smith (DNA 3:479-488, 1984). Thus, using the nucleotide and amino acid sequences of human wild-type HKI-18, one may introduce the alterations of choice. Additional techniques for generating variant-encoding nucleic acid sequences, vectors, methods for transfecting and culturing cells, methods of isolating proteins, methods of assessing sequence identity, methods of assessing amino acid sequence structural similarity, and other methods and compositions useful to the practice of this invention are described in International Patent Application WO 03/048185.

As indicated elsewhere herein, polypeptides and amino acid sequences of the invention can also comprise any number of suitable non-naturally occurring amino acid residues. Suitable non-naturally occurring amino acids include, without limitation, beta-alanine, desaminohistidine, trans-3-methylproline, 2,4-methanoproline, cis-4-hydroxyproline, trans-4-hydroxyproline, N-methylglycine, allo-threonine, methylthreonine, hydroxyethylcysteine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into polypeptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Polypeptides are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8,1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

As previously noted, nucleic acids encoding polypeptides of the invention include RNA and DNA polynucleotides. Methods for preparing DNA and RNA molecules are well known in the art. RNA can be isolated from a tissue or cell that produces large amounts of RNA (e.g., RNA encoding a polypeptide of the invention). Such tissues and cells can be identified by Northern blotting (Thomas, Proc. Natl. Acad. Sci. USA 77:5201, 1980), and include spinal cord, trachea, heart, colon, small intestine, and stomach tissues and cells. Total RNA can be prepared using guanidine-HCl extraction followed by isolation by centrifugation in a CsCl gradient (Chirgwin et al., Biochemistry 18:52-94,1979). Poly (A)⁺ RNA can be prepared from total RNA using the method of Aviv and Leder (Proc. Natl. Acad. Sci. USA 69:1408-12,1972). Complementary DNA (cDNA) can be prepared from poly (A)⁺ RNA using known methods. In the alternative, genomic DNA can be isolated. Polynucleotides encoding HKI-18 polypeptides can be identified and isolated by, for example, nucleic acid hybridization, or PCR techniques.

A full-length clone encoding a HKI-18 polypeptide can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to HKI-18 polypeptides, receptor fragments, or other specific binding partners.

The polynucleotides of the present invention can also be synthesized using automated equipment (“gene machines”). Gene synthesis methods are well known in the art. See, for example, Glick and Pasternak, Molecular Biotechnology, Principles & Applications of Recombinant DNA, ASM Press, Washington, D.C., 1994; Itakura et al., Annu. Rev. Biochem. 53: 323-356,1984; and Climie et al., Proc. Natl. Acad. Sci. USA 87:633-637, 1990.

The polynucleotide sequences encoding HKI-18 polypeptides disclosed herein can be used to isolate counterpart polynucleotides from other species (orthologs). These orthologous polynucleotides can be used, inter alia, to prepare the respective orthologous polypeptides. These other species include, but are not limited to mammalian, avian, amphibian, reptile, fish, insect and other vertebrate and invertebrate species. Of particular interest are polynucleotide sequences encoding HKI-18 polypeptides and HKI-18 polypeptides from other mammalian species, including murine, porcine, ovine, bovine, canine, feline, equine, and other primate polypeptides. Orthologs of human HKI-18 polypeptides can be cloned using information and compositions provided by the present invention in combination with conventional cloning techniques. For example, a cDNA can be cloned using mRNA obtained from a tissue or cell type that expresses HKI-18 polypeptides as disclosed herein. Suitable sources of mRNA can be identified by, probing Northern blots with probes designed from the sequences disclosed herein. A library is then prepared from mRNA of a positive tissue or cell line. A cDNA encoding HKI-18 polypeptides can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human HKI-18 polypeptides sequence disclosed herein. Within an additional method, the cDNA library can be used to transform or transfect host cells, and expression of the cDNA of interest can be detected with an antibody to HKI-18 polypeptide. Similar techniques can also be applied to the isolation of genomic clones.

Those skilled in the art will recognize that the sequence disclosed in SEQ ID NO:3 represents a single allele of the polynucleotide encoding human HKI-18 polypeptide and that natural variation, including allelic variation and alternative splicing, is expected to occur. Allelic variants of this sequence can be cloned by probing cDNA or genomic libraries from different individuals according to standard procedures. Allelic variants of the DNA sequence shown in SEQ ID NO:3, including those containing silent mutations and those in which mutations result in amino acid sequence changes, are within the scope of the present invention, as are polypeptides which are allelic variants of SEQ ID NO:1. cDNAs generated from alternatively spliced mRNAs, which retain the proteinase inhibiting activity of HKI-18 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

Nucleic acid (e.g., DNA) sequences encoding the HKI-18 polypeptide are usually inserted into a recombinant vector which may be any vector, which may conveniently be subjected to recombinant techniques, and the choice of vector will often depend on the host cell into which it is to be introduced. Thus, a vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

A vector in the context of the present invention is preferably an expression vector in which the DNA sequence encoding at least one HKI-18 polypeptide is operably linked to additional segments required or advantageous for expression of the polypeptide(s). An expression vector can be derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

A promoter may be any DNA sequence, which promotes transcriptional activity in the host cell of choice and may be derived from genes encoding polypeptides either homologous or heterologous to the host cell.

Examples of suitable promoters for directing the transcription of the DNA encoding the HKI-18 polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

DNA sequences encoding the HKI-18 polypeptide may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. A vector may also contain a set of RNA splice sites located downstream from the promoter and upstream from the insertion site for the polynucleotide sequence encoding the HKI-18 polypeptide itself. Preferred RNA splice sites may be obtained from adenovirus and/or immunoglobulin genes. Expression vectors also typically include a polyadenylation signal located downstream of the insertion site. Particularly preferred polyadenylation signals include the early or late polyadenylation signal from SV40 (Kaufman and Sharp, ibid.), the polyadenylation signal from the adenovirus 5 E1b region, the human growth hormone gene terminator (DeNoto et al. Nuc. Acids Res. 9:3719-3730, 1981) or the polyadenylation signal from the HKI-18 gene. Expression vectors may also include a noncoding viral leader sequence, such as the adenovirus 2 tripartite leader, located between the promoter and the RNA splice sites; and enhancer sequences, such as the SV40 enhancer.

A recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2g replication genes REP 1-3 and origin of replication.

A vector may also comprise a selectable marker, e.g. a gene the product of which complements a defect in the host cell, such as the gene coding for dihydrofolate reductase (DH FR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

For secretion from yeast cells, a secretory signal sequence may encode any signal peptide, which ensures efficient direction of the expressed HKI-18 polypeptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the HKI-18 polypeptide. The function of the leader peptide is to allow the expressed peptide to be directed from the endoplasmic reticulum to the Golgi apparatus and further to a secretory vesicle for secretion into the culture medium (i.e. exportation of the HKI-18 polypeptide across the cell wall or at least through the cellular membrane into the periplasmic space of the yeast cell). The leader peptide may be the yeast α-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the HKI-18 polypeptide, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Selectable markers may be introduced into the cell on a separate nucleic acid (e.g., separate plasmid) at the same time as the gene of interest, or they may be introduced on the same nucleic acid (e.g., the same plasmid or linear expression element). If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

Cells that have taken up target nucleic acid (e.g., target DNA), can be grown in an appropriate growth medium, typically 1-2 days, to begin expressing the gene of interest. As used herein the term “appropriate growth medium” means a medium containing nutrients and other components required for the growth of cells and the expression of the HKI-18 polypeptide of interest. Media generally include a carbon source, a nitrogen source, essential amino acids, essential sugars, vitamins, salts, phospholipids, protein, and growth factors. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the HKI-18 polypeptide of interest.

HKI-18 polypeptides can be tested for biological activity in protease inhibition assays, a variety of which are known in the art. Preferred assays include those measuring inhibition of trypsin, chymotrypsin, plasmin, cathepsin G, and human leukocyte elastase. See, for example, Petersen et al., Eur. J. Biochem. 235:310-316, 1996. In a typical procedure, the inhibitory activity of a test compound is measured by incubating the test compound with the proteinase, then adding an appropriate substrate, typically a chromogenic peptide substrate. See, for example, Norris et al. (Biol. Chem. Hoppe-Seyler 371:37-42, 1990). Briefly, various concentrations of the inhibitor are incubated in the presence of trypsin, plasmin, and plasma kallikrein in a low-salt buffer at pH 7.4, 25 degree. C. After 30 minutes, the residual enzymatic activity is measured by the addition of a chromogenic substrate (e.g., S2251 (D-Val-Leu-Lys-Nan) or S2302 (D-Pro-Phe-Arg-Nan), available from Kabi, Stockholm, Sweden) and a 30-minute incubation. Inhibition of enzyme activity is indicated by a decrease in absorbance at 405 nm or fluorescence Em at 460 nm. From the results, the apparent inhibition constant Kᵢ is calculated. The inhibition of coagulation factors (e.g., factor VIIa, factor Xa) can be measured using chromogenic substrates or in conventional coagulation assays (e.g., clotting time of normal human plasma; Dennis et al., ibid.).

HKI-18 polypeptides can be tested in animal models of disease, particularly tumor models, models of fibrinolysis, and models of imbalance of hemostasis. Suitable models are known in the art. For example, inhibition of tumor metastasis can be assessed in mice into which cancerous cells or tumor tissue have been introduced by implantation or injection (e.g., Brown, Advan. Enzyme Regul. 35:293-301,1995; Conway et al., Clin. Exp. Metastasis 14:115-124, 1996). Effects on fibrinolysis can be measured in a rat model wherein the enzyme batroxobin and radiolabeled fibrinogen are administered to test animals. Inhibition of fibrinogen activation by a test compound is seen as a reduction in the circulating level of the label as compared to animals not receiving the test compound. See, Lenfors and Gustafsson, Semin. Thromb. Hemost. 22:335-342, 1996. The effect on various states of systemic inflammatory response syndrome (ARDS, DIC) can be tested in animals treated with iv injection of LPS, heat-killed and/or live bacteria to induce sepsis (eg Taylor et al. Blood 91, 1609-1615, 1998; Welty-Wolf et al. Am Respir Crit Care Med 158; 610-619; 1998) HKI-18 polypeptides can be delivered to test animals by injection or infusion, or can be produced in vivo by way of, for example, viral or naked DNA delivery systems or transgenic expression.

Exemplary viral delivery systems include adenovirus, herpes virus, vaccinia virus, and adeno-associated virus (AAV) systems. Adenovirus, a double-stranded DNA virus, is currently the best studied gene transfer vector for delivery of heterologous nucleic acid (for a review, see Becker et al., Meth. Cell Biol. 43:161-189, 1994; and Douglas and Curiel, Science & Medicine 4:44-53,1997). The adenovirus system offers several advantages: adenovirus can (i) accommodate relatively large DNA inserts; (ii) be grown to high titer; (iii) infect a broad range of mammalian cell types; and (iv) be used with a large number of available vectors containing different promoters. Also, because adenoviruses are stable in the bloodstream, they can be administered by intravenous injection. By deleting portions of the adenovirus genome, larger inserts (up to 7 kb) of heterologous DNA can be accommodated. These inserts can be incorporated into the viral DNA by direct ligation or by homologous recombination with a co-transfected plasmid. In an exemplary system, the essential E1 gene is deleted from the viral vector, and the virus will not replicate unless the E1 gene is provided by the host cell (e.g., the human 293 cell line). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a signal sequence is present, secrete) the heterologous polypeptide. Secreted polypeptides will enter the circulation in the highly vascularized liver, and effects on the infected animal can be determined.

An alternative method of gene delivery comprises removing cells from the body and introducing a vector into the cells as a naked DNA plasmid. The transformed cells are then re-implanted in the body. Naked DNA vectors are introduced into host cells by methods known in the art, including transfection, electroporation, microinjection, transduction, cell fusion, DEAE dextran, calcium phosphate precipitation, use of a gene gun, or use of a DNA vector transporter. See, Wu et al., J. Biol. Chem. 263:14621-14624, 1988; Wu et al., J. Biol. Chem. 267:963-967, 1992; and Johnston and Tang, Meth. Cell Biol. 43:353-365, 1994.

Transgenic mice, engineered to express a HKI-18 gene, and mice that exhibit a complete absence of the function of the HKI-18 gene, referred to as “knockout mice” (Snouwaert et al., Science 257:1083, 1992), can also be generated by standard techniques (Lowell et al., Nature 366:740-742, 1993) and, accordingly, are another feature of the invention. These mice are employed to study the HKI-18 gene and the encoded polypeptide in an in vivo system. Transgenic mice are particularly useful for investigating the role of HKI-18 polypeptides in early development because they allow the identification of developmental abnormalities or blocks resulting from the over- or underexpression of a specific factor.

The HKI-18 polypeptides of the present invention, including full-length polypeptides, biologically active fragments, and related fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., N.Y., 1987.

In general, a DNA sequence encoding a HKI-18 polypeptide can be operably linked to other genetic elements required for its expression, generally including a transcription promoter and terminator, within an expression vector. The vector also commonly contains one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a HKI-18 polypeptide into the secretory pathway of a host cell, a secretory signal sequence (also known as a leader sequence, prepro sequence or pre sequence) is provided in the expression vector. The secretory signal sequence may be that of the HKI-18 polypeptide, or may be derived from another secreted polypeptide (e.g., t-PA; see, U.S. Pat. No. 5,641,655) or synthesized de novo. The secretory signal sequence is operably linked to the DNA sequence encoding a HKI-18 polypeptide, i.e., the two sequences can be joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells can be suitable host cells. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., ibid.), and liposome-mediated transfection (Hawley-Nelson et al., Focus 15:73, 1993; Ciccarone et al., Focus 15:80, 1993). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72,1977), Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), DUKX cells (Urlaub and Chasin, Proc. Natl. Acad. Sci. USA 77:4216-4220,1980) and Chinese hamster ovary (e.g. CHO-K1; ATCC No. CCL 61) cell lines.

Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter. Expression vectors for use in mammalian cells include pZP-1 and pZP-9, which have been deposited with the American Type Culture Collection, Rockville, Md. USA under accession numbers 98669 and 98668, respectively.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. The use of Agrobacterium rhizogenes as a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463.

Insect cells can be infected with recombinant baculovirus, commonly derived from Autographa californica nuclear polyhedrosis virus (AcNPV). See, King and Possee, The Baculovirus Expression System: A Laboratory Guide, London, Chapman & Hall; O'Reilly et al., Baculovirus Expression Vectors: A Laboratory Manual, New York, Oxford University Press., 1994; and Richardson, Ed., Baculovirus Expression Protocols. Methods in Molecular Biology, Humana Press, Totowa, N.J., 1995. Recombinant baculovirus can also be produced through the use of a transposon-based system described by Luckow et al. (J. Virol. 67:4566-4579,1993). This system, which utilizes transfer vectors, is commercially available in kit form (Bac-to-Bac.™. kit; Life Technologies, Rockville, Md.). The transfer vector (e.g., pFastBacl.™.; Life Technologies) contains a Tn7 transposon to move the DNA encoding the polypeptide of interest into a baculovirus genome maintained in E. coli as a large plasmid called a “bacmid.” See, Hill-Perkins and Possee, J. Gen. Virol. 71:971-976, 1990; Bonning et al., J. Gen. Virol. 75:1551-1556, 1994; and Chazenbalk and Rapoport, J. Biol. Chem. 270:1543-1549, 1995. In addition, transfer vectors can include an in-frame fusion with DNA encoding a polypeptide extension or affinity tag as disclosed above. Using techniques known in the art, a transfer vector containing a polynucleotide sequence encoding the HKI-18 polypeptide is transformed into E. coli host cells, and the cells are screened for bacmids which contain an interrupted lacZ gene indicative of recombinant baculovirus. The bacmid DNA containing the recombinant baculovirus genome is isolated, using common techniques, and used to transfect Spodoptera frugiperda cells, such as Sf9 cells. Recombinant virus that expresses HKI-18 polypeptide is subsequently produced. Recombinant viral stocks are made by methods commonly used the art.

For polypeptide production, the recombinant virus is used to infect host cells, typically a cell line derived from the fall armyworm, Spodoptera frugiperda (e.g., Sf9 or Sf21 cells) or Trichoplusia ni (e.g., High Five.™. cells; Invitrogen, Carlsbad, Calif.). See, in general, Glick and Pasternak, Molecular Biotechnology: Principles and Applications of Recombinant DNA, ASM Press, Washington, D.C., 1994. See also, U.S. Pat. No. 5,300,435. Serum-free media are used to grow and maintain the cells. Suitable media formulations are known in the art and can be obtained from commercial suppliers. The cells are grown up from an inoculation density of approximately 2-5×10⁵ cells to a density of 1-2×10⁶ cells, at which time a recombinant viral stock is added at a multiplicity of infection (MOI) of 0.1 to 10, more typically near 3. Procedures used are generally described in available laboratory manuals (e.g., King and Possee, ibid.; O'Reilly et al., ibid.; Richardson, ibid.).

Fungal cells, including yeast cells, can also be used within the present invention. Yeast species of particular interest in this regard include Saccharomyces cerevisiae, Saccharomyces kluyveri, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311; Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pats. Nos. 4,990,446; 5,063,154; 5,139,936 and 4,661,454.

Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-3465,1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533. The use of Pichia methanolica as host for the production of recombinant polypeptides is disclosed in U.S. Pats. No. 5,716,808, 5,736,383, 5,854,039, and 5,888,768; and WIPO Publications WO 97/17450 and WO97/17451.

Prokaryotic host cells, including strains of the bacteria Escherichia coli, Bacillus and other genera are also useful host cells within the present invention. Techniques for transforming these hosts and expressing foreign DNA sequences cloned therein are well known in the art (see, e.g., Sambrook et al., ibid.). When expressing a HKI-18 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the polypeptide, thereby obviating the need for denaturation and refolding. Methods for producing heterologous disulfide bond-containing polypeptides in bacterial cells are disclosed by Georgiou et al., U.S. Pat. No. 6,083,715.

Transformed or transfected host cells can be cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

HKI-18 polypeptides can also be prepared through chemical synthesis according to methods known in the art, including exclusive solid phase synthesis, partial solid phase methods, fragment condensation or classical solution synthesis. See, for example, Merrifield, J. Am. Chem. Soc. 85:2149, 1963; Stewart et al., Solid Phase Peptide Synthesis (2nd edition), Pierce Chemical Co., Rockford, Ill., 1984; Bayer and Rapp, Chem. Pept. Prot. 3:3, 1986; and Atherton et al., Solid Phase Peptide Synthesis: A Practical Approach, IRL Press, Oxford, 1989.

It is preferred to purify the polypeptides of the present invention to about 80% purity, more preferably to about 90% purity, even more preferably about 95% purity, and particularly preferred is a pharmaceutically pure state, that is greater than 99.9% pure with respect to contaminating macromolecules, particularly other polypeptides and nucleic acids, and such polypeptides also are desirably free of any detectable infectious and/or pyrogenic agents. Preferably, a purified polypeptide is substantially free of other polypeptides, particularly other polypeptides of animal origin.

HKI-18 polypeptides can be purified by conventional protein purification methods, typically by a combination of chromatographic techniques. Polypeptides comprising a polyhistidine affinity tag (typically about 6 histidine residues) are purified by affinity chromatography on a nickel chelate resin. See, for example, Houchuli et al., Bio/Technol. 6: 1321-1325,1988.

Using methods known in the art, HKI-18 polypeptides can be produced glycosylated or non-glycosylated; PEGylated or non-PEGylated; and may or may not include an initial methionine amino acid residue.

The HKI-18 polypeptides of the invention can be used in the treatment and/or prevention of conditions associated with excessive proteinase activity, and, in particular aspects, for the treatment and/or prevention (as that is described elsewhere herein) with an excess of trypsin, plasmin, kallikrein, elastase, cathepsin G, proteinase-3, thrombin, factor VIIa, factor IXa, factor Xa, factor XIa, factor XIIa, or matrix metalloproteinases. Such conditions include, but are not limited to, acute pancreatitis, cardiopulmonary bypass (CPB)-induced pulmonary injury, allergy-induced protease release, deep vein thrombosis, myocardial infarction, shock (including septic shock), hyperfibrinolytic hemorrhage, emphysema, rheumatoid arthritis, adult respiratory distress syndrome, chronic inflammatory bowel disease, psoriasis, systemic inflammatory response syndrome and other inflammatory conditions. HKI-18 polypeptides are also contemplated for use in preservation of platelet function, organ preservation, and in promoting, enhancing, and/or inducing wound healing.

HKI-18 polypeptides may also be useful in the treatment of conditions arising from an imbalance in hemostasis, including acquired coagulopathies, primary fibrinolysis and fibrinolysis due to cirrhosis, and complications from high-dose thrombolytic therapy. Acquired coagulopathies can result from liver disease, uremia, acute disseminated intravascular coagulation, post-cardiopulmonary bypass, massive transfusion, or Warfarin overdose (Humphries, Transfusion Medicine 1:1181-1201,1994). A deficiency or dysfunction in any of the procoagulant mechanisms predisposes the patient to either spontaneous hemorrhage or excess blood loss associated with trauma or surgery. Acquired coagulopathies usually involve a combination of deficiencies, such as deficiencies of a plurality of coagulation factors, and/or platelet dysfunction. In addition, patients with liver disease commonly experience increased fibrinolysis due to an inability to maintain normal levels of alpha 2-antiplasmin and/or decreased hepatic clearance of plasminogen activators (Shuman, Hemorrhagic Disorders, in Bennet and Plum, eds. Cecil Textbook of Medicine, 20th ed., W. B. Saunders Co., 1996). Primary fibrinolysis results from a massive release of plasminogen activator. Conditions associated with primary fibrinolysis include carcinoma of the prostate, acute promyelocytic leukemia, hemangiomas, and sustained release of plasminogen activator by endothelial cells due to injection of venoms. The condition becomes critical when enough plasmin is activated to deplete the circulating level of α2-antiplasmin (Shuman, ibid.). Data suggest that plasmin on endothelial cells may be related to the pathophysiology of bleeding or rethrombosis observed in patients undergoing high-dose thrombolytic therapy for thrombosis. Plasmin may cause further damage to the thrombogenic surface of blood vessels after thrombolysis, which may result in rethrombosis (Okajima, J. Lab. Clin. Med. 126:1377-1384,1995).

Additional antithrombotic uses of HKI-18 polypeptides include treatment or prevention of deep vein thrombosis, pulmonary embolism, and post-surgical thrombosis.

HKI-18 polypeptides may also be used within methods for inhibiting blood coagulation in mammals, such as in the treatment of disseminated intravascular coagulation. HKI-18 polypeptides may thus be used in place of known anticoagulants such as heparin, coumarin, and anti-thrombin III. Such methods will generally include administration of the polypeptide in an amount sufficient to produce a clinically significant inhibition of blood coagulation. Such amounts will vary with the nature of the condition to be treated, but can be predicted on the basis of known assays and experimental animal models, and will in general be within the ranges disclosed below.

HKI-18 polypeptides may also find therapeutic use in the blockage of proteolytic tissue degradation. Proteolysis of extracellular matrix, connective tissue, and other tissues and organs is an element of many diseases. This tissue destruction is believed to be initiated when plasmin activates one or more matrix metalloproteinases (e.g., collagenase and metalloelastases). Inhibition of plasmin by HKI-18 polypeptides may thus be beneficial in the treatment of these conditions.

Matrix metalloproteinases (MMPs) are believed to play a role in metastases of cancers, abdominal aortic aneurysm, multiple sclerosis, rheumatoid arthritis, osteoarthritis, trauma and hemorrhagic shock, and cornial ulcers. MMPs produced by tumor cells break down and remodel tissue matrices during the process of metastatic spread. There is evidence to suggest that MMP inhibitors may block this activity (Brown, Advan. Enzyme Regul. 35:293-301, 1995). Abdominal aortic aneurysm is characterized by the degradation of extracellular matrix and loss of structural integrity of the aortic wall. Data suggest that plasmin may be important in the sequence of events leading to this destruction of aortic matrix (Jean-Claude et al., Surgery 116:472-478, 1994). Proteolytic enzymes are also believed to contribute to the inflammatory tissue damage of multiple sclerosis (Gijbels, J. Clin. Invest. 94:2177-2182,1994). Rheumatoid arthritis is a chronic, systemic inflammatory disease predominantly affecting joints and other connective tissues, wherein proliferating inflammatory tissue (panus) may cause joint deformities and dysfunction (see, Arnett, in Cecil Textbook of Medicine, ibid.). Osteoarthritis is a chronic disease causing deterioration of the joint cartilage and other joint tissues and the formation of new bone (bone spurs) at the margins of the joints. There is evidence that MMPs participate in the degradation of collagen in the matrix of osteoarthritic articular cartilage. Inhition of MMPs results in the inhibition of the removal of collagen from cartilage matrix (Spirito, Inflam. Res. 44 (supp. 2):S131-S132, 1995; O'Byrne, Inflam. Res. 44 (supp. 2):S117-S118, 1995; Karran, Ann. Rheumatic Disease 54:662-669, 1995). HKI-18 polypeptides may also be useful in the treatment of trauma and hemorrhagic shock. Data suggest that administration of an MMP inhibitor after hemorrhage improves cardiovascular response, hepatocellular function, and microvascular blood flow in various organs (Wang, Shock 6:377-382, 1996). Corneal ulcers, which can result in blindness, manifest as a breakdown of the collagenous stromal tissue. Damage due to thermal or chemical injury to corneal surfaces often results in a chronic wound-healing situation. There is direct evidence for the role of MMPs in basement membrane defects associated with failure to re-epithelialize in cornea or skin (Fini, Am. J. Pathol. 149:1287-1302,1996).

The HKI-18 polypeptides of the present invention may be combined with other therapeutic agents to augment the activity (e.g., antithrombotic or anticoagulant activity) of such agents. For example, a HKI-18 polypeptide may be used in combination with tissue plasminogen activator in thrombolytic therapy. Compositions comprising such combinations of agents and methods of co-administering such agents (whether simultaneously or in a progression) are additional aspects of the invention.

The polypeptides and/or compositions of the invention can be administered in any suitable dose for inducing, promoting, and/or enhancing the desired physiological response(s). The dosage will vary with a number of factors that generally are within the scope of routine experimentation for those of ordinary skill in the art. For example, the dosage of HKI-18 polypeptides can vary according to the severity of the condition being treated. Typically, a suitable dosage for such polypeptides will range from approximately 0.01 mg/kg to about 10 mg/kg body weight, such as about 0.1 mg/kg to about 5 mg/kg, typically about 0.1 mg/kg to about 1 mg/kg. The polypeptides are typically formulated in a pharmaceutically acceptable carrier, vehicle, diluent, and/or excipient. In one exemplary aspect, polypeptides of the invention are formulated in a form suitable for injection or infusion, such as by dilution with sterile water, an isotonic saline or glucose solution, or similar vehicle. In the alternative, the polypeptide may be packaged as a lyophilized powder, optionally in combination with a pre-measured diluent, and resuspended immediately prior to use. Pharmaceutical compositions may further include one or more excipients, preservatives, solubilizers, buffering agents, albumin to prevent protein loss on vial surfaces, etc. Formulation methods are within the level of ordinary skill in the art. See, Remington: The Science and Practice of Pharmacy, Gennaro, ed., Mack Publishing Co., Easton, Pa., 19th ed., 1995.

Gene therapy provides an alternative therapeutic approach for delivery of HKI-18 polypeptides to a subject. If a mammal has a mutated or absent HKI-18 gene, a nucleic acid encoding a HKI-18 polypeptide can be introduced into the cells of the mammal (either as a naked nucleic acid molecule or by way of a suitable vector). In one aspect the invention relates to method for introduction of a HKI-18 gene into a mammal. In one aspect, a HKI-18 gene is introduced in vivo in a viral vector. Such vectors include an attenuated or defective DNA virus, such as herpes simplex virus (HSV), papillomavirus, Epstein Barr virus (EBV), adenovirus, adeno-associated virus (AAV), and the like. Defective viruses, which entirely or almost entirely lack viral genes, are often preferred gene delivery vehicles. A defective virus is not infective after introduction into a cell. Use of defective viral vectors allows for administration to cells in a specific, localized area, without concern that the vector can infect other cells. Examples of particular vectors include, without limitation, a defective herpes simplex virus 1 (HSV1) vector (Kaplitt et al., Molec. Cell. Neurosci. 2:320-30,1991); an attenuated adenovirus vector, such as the vector described by Stratford-Perricaudet et al., J. Clin. Invest. 90:626-30,1992; and a defective adeno-associated virus vector (Samulski et al., J. Virol. 61:3096-101, 1987; Samulski et al., J. Virol. 63:3822-8,1989). Within another aspect, a HKI-18 gene can be introduced in a retroviral vector, as described, for example, by Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; Dougherty et al., WIPO Publication No. WO 95/07358; and Kuo et al., Blood 82:845, 1993. Alternatively, the vector can be introduced by lipofection in vivo using any suitable type of liposomes. Synthetic cationic lipids can be used to prepare liposomes for in vivo transfection of a gene encoding a marker (Feigner et al., Proc. Natl. Acad. Sci. USA 84:7413-7, 1987; Mackey et al., Proc. Natl. Acad. Sci. USA 85:8027-31, 1988). Within a further aspect, target cells are removed from the body, and a vector is introduced into the cells as a naked DNA plasmid. The transformed cells are then re-implanted into the body. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, for example, Wu et al., J. Biol. Chem. 267:963-7,1992; Wu et al., J. Biol. Chem. 263:14621-4,1988.

HKI-18 polypeptides can also be used to prepare antibodies that specifically bind to HKI-18 polypeptides. As used herein, the term “antibodies” includes polyclonal antibodies, monoclonal antibodies, antigen-binding fragments thereof such as F(ab′)₂ and Fab fragments, single chain antibodies, and the like, including genetically engineered antibodies. Non-human antibodies can be humanized by grafting only non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced. One skilled in the art can generate humanized antibodies with specific and different constant domains (i.e., different Ig subclasses) to facilitate or inhibit various immune functions associated with particular antibody constant domains. Alternative techniques for generating or selecting antibodies useful herein include in vitro exposure of lymphocytes to a HKI-18 polypeptide, and selection of antibody display libraries in phage or similar vectors (for instance, through use of immobilized or labeled HKI-18 polypeptide). Antibodies are defined to be specifically binding if they bind to a HKI-18 polypeptide with an affinity at least 10-fold greater than the binding affinity to control (non-HKI-18) polypeptide. It is preferred that the antibodies exhibit a binding affinity (Kₐ) of 10⁶ M ⁻¹ or greater, preferably 10⁷ M⁻¹ or greater, more preferably 10⁸ M⁻¹ or greater, and most preferably 10⁹ M⁻¹ or greater. The affinity of a monoclonal antibody can be readily determined by one of ordinary skill in the art (see, for example, Scatchard, Ann. NY Acad. Sci. 51: 660-672, 1949). Such antibodies are another feature of the invention.

Methods for preparing polyclonal and monoclonal antibodies are well known in the art (see for example, Hurrell, J. G. R., Ed., Monoclonal Hybridoma Antibodies: Techniques and Applications, CRC Press, Inc., Boca Raton, Fla., 1982). As would be evident to one of ordinary skill in the art, polyclonal antibodies can be generated from a variety of warm-blooded animals such as horses, cows, goats, sheep, dogs, chickens, rabbits, mice, and rats. The immunogenicity of a HKI-18 polypeptide may be increased through the use of an adjuvant such as alum (aluminum hydroxide) or Freund's complete or incomplete adjuvant. Polypeptides useful for immunization also include fusion polypeptides, such as fusions of a HKI-18 polypeptide or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

Immunogenic fragments of HKI-18 polypeptides may be as small as 5 residues. It is preferred to use polypeptides that are hydrophilic or comprise a hydrophilic region. A variety of assays known to those skilled in the art can be utilized to detect antibodies that specifically bind to a HKI-18 polypeptide. Exemplary assays are described in detail in Antibodies: A Laboratory Manual, Harlow and Lane (Eds.), Cold Spring Harbor Laboratory Press, 1988. Representative examples of such assays include concurrent immunoelectrophoresis, radio-immunoassays, radio-immunoprecipitations, enzyme-linked immunosorbent assays (ELISA), dot blot assays, Western blot assays, inhibition or competition assays, and sandwich assays.

Antibodies to HKI-18 polypeptides may be used for affinity purification of HKI-18 polypeptides; within diagnostic assays for determining circulating levels of HKI-18 polypeptides; for detecting or quantitating soluble HKI-18 polypeptide as a marker of underlying pathology or disease; for immunolocalization within whole animals or tissue sections, including immunodiagnostic applications; for immunohistochemistry; for screening expression libraries; and for other uses that will be evident to those skilled in the art. For certain applications, including in vitro and in vivo diagnostic uses, it is advantageous to employ labeled antibodies. Suitable direct tags or labels include radionuclides, enzymes, substrates, cofactors, inhibitors, fluorescent markers, chemiluminescent markers, magnetic particles and the like; indirect tags or labels may feature use of biotin-avidin or other complement/anti-complement pairs as intermediates.

HKI-18 polypeptides may be used in the laboratory or in commercial preparations of polypeptides from cultured cells. The polypeptides can be used alone to inhibit specific proteolysis or can be combined with other proteinase inhibitors to provide a “cocktail” with a broad spectrum of activity. Of particular interest is the inhibition of cellular proteases, which can be released during cell lysis. The polypeptides can also be used in the laboratory as a tissue culture additive to prevent cell detachment.

The present invention also provides reagents for use in diagnostic applications. For example, the HKI-18 gene, a probe comprising DNA or RNA or a subsequence thereof encoding the HKI-18 polypeptide can be used to determine if the HKI-18 gene is present on chromosome 7 or if a mutation has occurred. Detectable chromosomal aberrations at the locus of the HKI-18 gene include, but are not limited to, aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, short tandem repeat (STR) analysis employing PCR techniques, and other genetic linkage analysis techniques known in the art (Sambrook et al., ibid.; Ausubel et. al., ibid.; Marian, Chest 108:255-65, 1995).

Polynucleotides encoding HKI-18 polypeptides can also be used for chromosomal mapping. Localization of the HKI-18 gene facilitates the establishment of directly proportional physical distances between newly discovered genes of interest and previously mapped markers, including the HKI-18 gene. The precise knowledge of a gene's position can be useful for a number of purposes, including: 1) determining if a newly identified sequence is part of an previously identified gene or gene segment and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have. A useful technique in this regard is radiation hybrid mapping, a somatic cell genetic technique developed for constructing high-resolution, contiguous maps of mammalian chromosomes (Cox et al., Science 250:245-50, 1990). Partial or full knowledge of a gene's sequence allows one to design PCR primers suitable for use with chromosomal radiation hybrid mapping panels. Radiation hybrid mapping panels, which are commercially available (e.g., the Stanford G3 RH Panel and the GeneBridge 4 RH Panel, available from Research Genetics, Inc., Huntsville, Ala.), cover the entire human genome. These panels enable rapid, PCR-based chromosomal localizations and ordering of genes, sequence-tagged sites (STSs), and other nonpolymorphic and polymorphic markers within a region of interest.

Sequence tagged sites (STSs) can also be used independently for chromosomal localization. An STS is a DNA sequence that is unique in the human genome and can be used as a reference point for a particular chromosome or region of a chromosome. An STS is defined by a pair of oligonucleotide primers that are used in a polymerase chain reaction to specifically detect this site in the presence of all other genomic sequences. Since STSs are based solely on DNA sequence they can be completely described within an electronic database (for example, Database of Sequence Tagged Sites (dbSTS), GenBank; National Center for Biological Information, National Institutes of Health, Bethesda, Md.; http://www.ncbi.nlm.nih.gov) and can be searched with a gene sequence of interest for the mapping data contained within these short genomic landmark STS sequences.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### Example 1

Cloning of Human Wild Type HKI-18

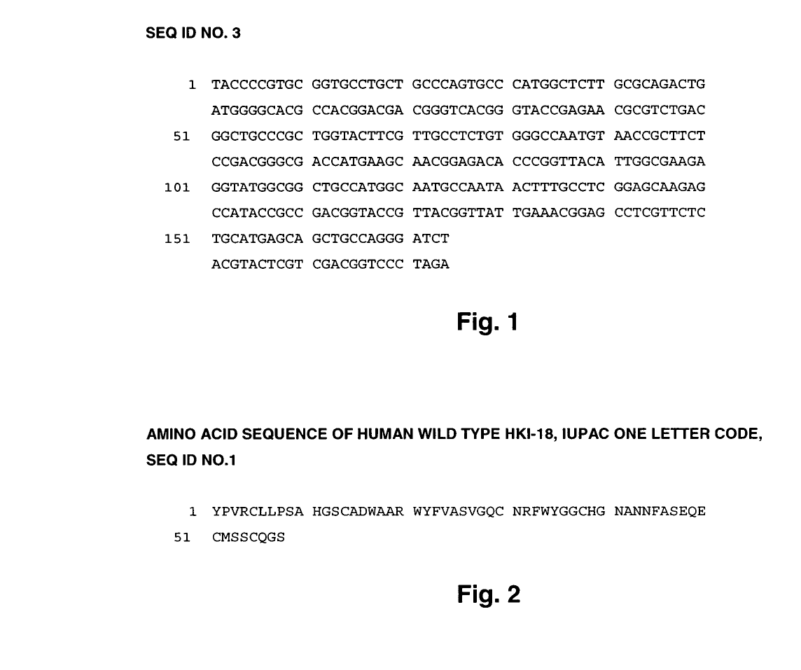

Human wild type HKI-18 is included in a 133.5 kDa hypothetical polypeptide, which has been predicted from the DNA sequence EMBL:AF109907. The DNA sequence encoding the Kunitz domain is seen in FIG. 1, the amino acid sequence is seen in FIG. 2.

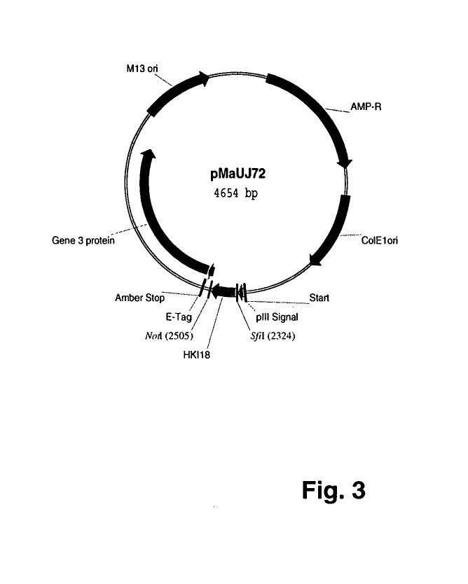

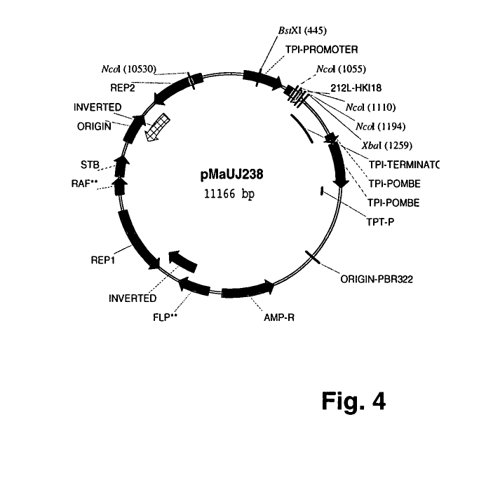

The DNA encoding the human wild type HKI-18 domain was amplified by a polymerase chain reaction (PCR) in which human testis first-strand cDNA (Clontech, Multiple Tissue cDNA Panels) was used as template. 5 units Herculase Polymerase (Stratagene) and 1 nmol of the primers oMaUJ41 and oMaUJ42 (Table 3) were used in a 100 μl reaction. oMaUJ41 anneals to 19 bp in the 5′ end of the human wild type HKI-18 open reading frame and contains an SfiI site in a 5′ extension. oMaUJ42 anneals to 22 bp in the 3′end of the human wild type HKI-18 open reading frame and contains an NotI site in a 5′extension. The PCR reaction was carried out as follows: 94° C. for 2 min, 4 cycles of 94° C. for 1 min; 60° C. 1 min; 72° C. for 30 sec, 4 cycles of 94° C. for 1 min; 55° C. 1 min; 72° C. for 30 sec; 25 cycles of 94° C. for 1 min; 50° C. 1 min; 72° C. for 30 sec. The resulting PCR product was digested with SfiI/NotI and cloned in the SfiI/NotI site of pCANTAB 5E (Amersham Pharmacia), resulting in pMaUJ72 (FIG. 3). pMaUJ72 was checked for insert by appropriate restriction nucleases (e.g. SfiI and NotI) and was shown by sequence analysis using the primer oMaUJ11 (Table 3) to contain the proper sequence of human wild type HKI-18.

Construction of 212L-HKI18 Fusion

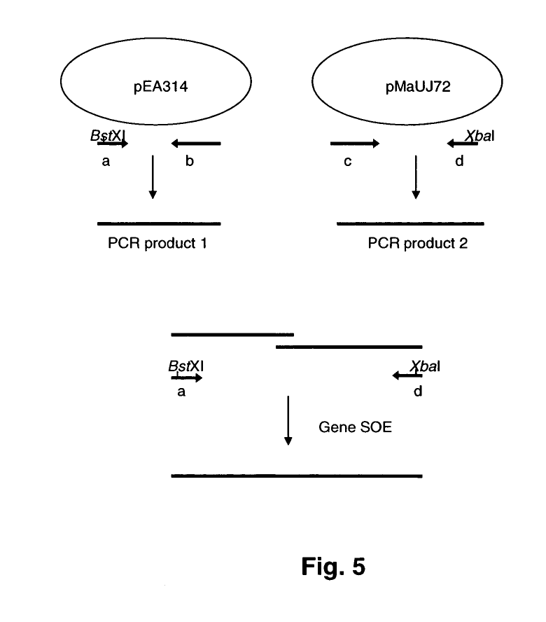

The 212L-HKI18 construction, called pMaUJ238 (FIG. 4), was created by gene splicing by overlap extension (gene SOE) (Horton et al., Gene 77, 61-68, 1989), as illustrated in FIG. 5. 5 units Taq polymerase (Promega) was used in 100 μl reactions. The primers and templates used in the construction of 212-HKI18 are listed in table 2 and described in the following.

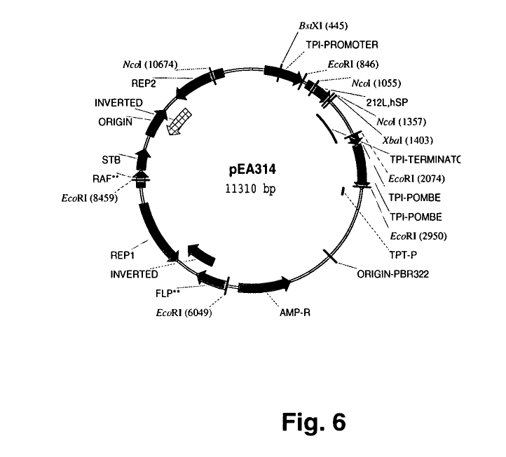

pEA314 is identical to pKFN-1847 (Thim L. et al. FEBS 318, 345-352, 1993) except for a single nucleotide mutation changing Asn₉₉ of human spasmolytic polypeptide (hSP) to Lys₉₉. pEA314 (FIG. 6) contains an expression cassette comprising an EcoRI-XbaI fragment inserted between the transcription-promoter and the transcription-terminator of the S. cerevisiae TPI gene. The EcoRI-XbaI fragment encodes a fusion product composed of the 212L leader sequence, a Lys-Arg cleavage site for the dibasic processing endopeptidase KEX2, and the mutant hSP-Lys₉₉.

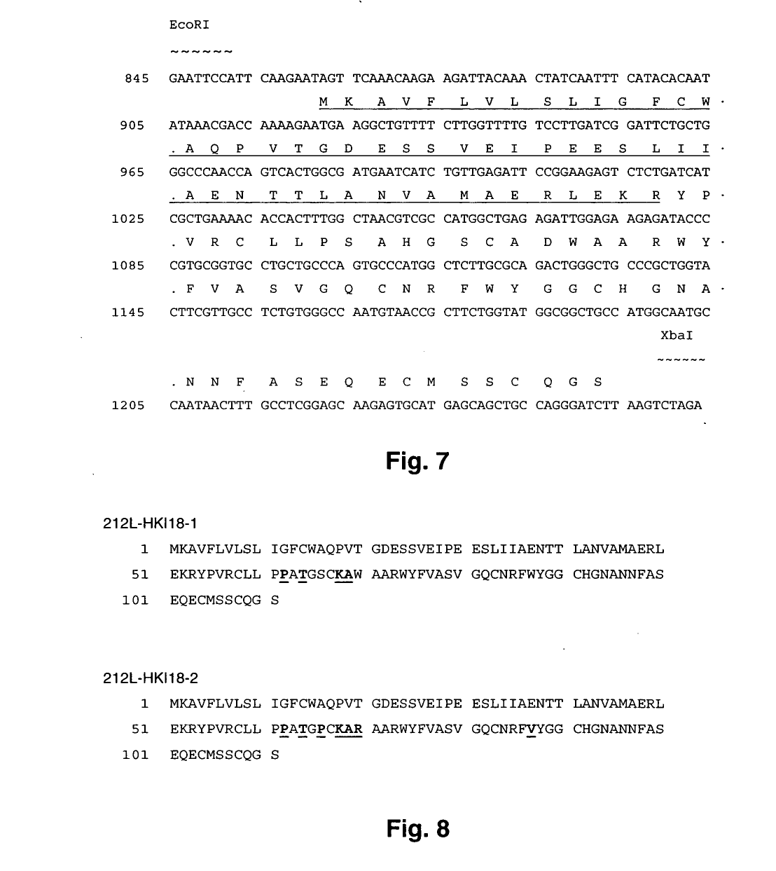

The 5′ end of primer b (corresponding to oMaUJ88) is complementary to the 3′ end of primer c (corresponding to oMaUJ89). In the gene SOE reaction (FIG. 5) the two independent PCR products 1 and 2 are incubated with the primers a (corresponding to oMaUJ87) and d (corresponding to oMaUJ90). PCR product 1 includes the 212L leader sequence and the Lys-Arg Kex2p cleavage site. PCR product 2 contains the HKI18 ORF. The 5′ end of oMaUJ87 contains a BstXI site, the 5′ end of oMaUJ90 contains an XbaI site. The resulting PCR product from the gene SOE reaction was digested with BstXI and XbaI and ligated to pEA314 digested with BstXI and XbaI. The final construct called pMaUJ238 (FIG. 4) was propagated in E. coli, grown in the presence of ampicillin and isolated using standard techniques (Sambrook et al., Molecular cloning. A laboratory manual. Cold Spring Harbor Laboratory. Cold Spring Harbor, N.Y., 1989). pMaUJ238 was checked for insert by appropriate restriction nucleases (e.g. NcoI, BstXI and XbaI) and was shown by sequence analysis using primers oMaUJ125 or oMaUJ126 (Table 3) to contain the proper construct of 212L-HKI18 (FIG. 7).

Mutants of HKI-18 Polypeptides

The mutants 212L-HKI18-1 (212L-HKI18 P9, T11, K15, A16) and 212L-HKI18-2 (212L-HKI18 P9, T11, P13, K15, A16, R17, V34) (FIG. 8), were created by assembly PCR (Stemmer et al., Gene 164, 49-53, 1995). The DNAs covering the mutated sequence were synthesized from oligos 40-45 bp in length, using High Fidelity polymerase (Roche).

The PCR reaction was carried out as follows: 55 cycles of 94° C. in 30 sec; 52° C. in 30 sec; 72° C. 30 sec, followed by addition of the two outside primers oMaUJ224 and oMaUJ233 (Table 3) and 15 cycles of 94° C. in 30 sec; 50° C. in 30 sec; 72° C. 30 sec. The oligonucleotides used for assembly PCR introduce the four amino acid substitutions in HKI18-1 (oMaUJ224-233) and the 7 amino acid substitutions in HKI18-2 (oMaUJ224, 225, 228 and oMaUJ231-237), respectively. Furthermore, a silent mutation in oMaUJ224 removes the NcoI site, which is present in the 212L leader sequence.

The 5′ end of oMaUJ224 contains an NcoI site, and the 5′ end of oMaUJ233 contains an XbaI site. The resulting PCR products were digested with NcoI and XbaI and ligated into the NcoI/XbaI site of pEA314 (FIG. 6) by partial digestions. The resulting yeast plasmids pMaUJ365 and pMaUJ367 expressing HKI18-1 and HKI18-2, respectively, were propagated in E. coli, grown in the presence of ampicillin and isolated using standard techniques (Sambrook et al., 1989). pMaUJ365 and pMaUJ367 were checked for insert by appropriate restriction nucleases (e.g. NcoI and XbaI) and are shown by sequence analysis using primers oMaUJ125 or oMaUJ126 (Table 3) to contain the proper constructs of 212L-HKI18-1 and 212L-HKI18-2, respectively.

Expression of HKI-18 Polypeptides in Yeast

The plasmids are transformed into S. cerevisiae strain MT663 (MATa/MATα pep4-3/pep4-3 HIS4/his4 tpi::LEU2/tpi::LEU2 Cir⁺). Strain MT663 was deposited in the Deutsche Sammlung von Mikroorganismen und Zellkulturen in connection with filing WO 92/11378 and was given the deposit number DSM 6278. Transformation of MT633 is conducted as described in WO 98/01535.

Yeast transformants harboring pMaUJ238, pMaUJ365 and pMaUJ367, respectively, are selected by glucose utilization as carbon source on YPD (1% yeast extract, 2% peptone, 2% glucose) agar (2%) plates. The transformants yMaUJ33, yMaUJ69 and yMaUJ70 (Table 4) are selected for fermentation.

Yeast strains yMaUJ33, yMaUJ69 and yMaUJ70 are cultivated for 72 hours in ZYM media (2% yeast extract, 1% peptone, 6% glucose), and the cultures are pooled resulting in a final OD₆₀₀ of approximately 15-20. After centrifugation the cell pellet is discarded and the supernatant is used for purification and characterization of the HKI-18 polypeptide.

Purification of HKI-18 Polypeptides

The supernatant is adjusted to pH 7 and centrifuged to separate the cells. The supernatant is adjusted to pH 3 and centrifuged to a clarified fraction at 10000 g, 30 minutes, 4° C. on Sorwall SLA. Two identical runs are performed on a C4 column: 350 ml liquid is applied on a C4 column (Jupiter 15 μm 300 Å 10×250 mm) which equilibrated with 20 mM Citric acid 50 mM KCl pH 3.0. After wash with equilibration buffer a gradient elution is performed with increasing EtOH-concentration up to 60%. The fractions are analyzed by SDS, HKI18 elutes at about 30% EtOH.

The pool is collected and the buffer is exchanged on a SephadexG25 (HiPrep 26/10) to 20 mM Na-phosphate pH 7.2. At least a fraction is concentrated on a Centriprep with 3 kDa cut off.

HKI-18 polypeptides may alternatively be made synthetic by methods known to the person skilled in the art.

Characterization of HKI18

The concentrated sample is analyzed by Mass-spectroscopy, SDS, Sequence analysis, Enzyme activity and HPLC.

Activity assay:

Inhibition of selected proteases by HKI-18 polypeptides is measured essentially as described by Petersen et al. FEBS Letters 338 53-57,1994. Briefly, 25 μl Trypsin (50 nM) or plasmin (50 nM) was mixed with 25 μl HKI-18 analogues or aprotinin dilutions and 25 μl 20 mM CaCl₂. The mixture was incubated 15 min at room temperature before 25 μl 2 mM S-2222 or S-2251 respectively was added. The reaction was stopped after 60 min with 50 μl 1 M citrate buffer and the absorbance read at 405 nm. Assay buffer: 50 mM TRIS, 100 mM NaCl, 0.1% BSA, pH 7.4.

Chromogenic substrates H-D-Val-Leu-Lys-pNA; MeO-Suc-Arg-Pro-Tyr-pNA; H-D-Val-Leu-Arg-pNA; Glu-Gly-Arg-pNA; H-D-Ile-Pro-Arg-pNA; <Glu-Pro-Arg-pNA; H-D-Pro-Phe-Arg-PNA; H-D-Phe-Pip-Arg-pNA; Cbo-D-Arg-Gly-Arg-pNA are purchased from Chromogenix (Moelndal, Sweden), MeO-CO-CHA-Gly-Arg-pNA from NycoMed (Oslo, Norway), and MeO-Suc-Ala-Ala-Pro-Val-pNA; Suc-Ala-Ala-Pro-Phe-pNA from Sigma (St. Louis, Mo, USA).

Trypsin, chymotrypsin, thrombin, plasmin, glandular kallikrein, human tissue-type plasminogen activator (tPA), activated protein C (APC) are from Sigma (St. Louis, Mo. USA) uPA are from FLUKA (Milwaukee, Wis., USA). Recombinant factor VII (FVIIa) is from Novo Nordisk (Bagsvaerd, Denmark). Human leukocyte elastase (HLE) and cathepsin G (CatG) were purified by previously described procedures (Baugh, R. J. and Travis, J. (1976) Biochemistry 15 836-843). Activated factor X (FXa), activated factor XII (FXIIa) and recombinant tissue factor (TF) are from Calbiochem-Novabiochem Corporation (San Diego, Calif. USA).

Measurements of protease inhibition are performed by incubation with HKI-18 polypeptide for 30 min and subsequent substrate addition. Residual activity is then measured. The reaction takes place in microtiter wells in 100 mM NaCl, 50 mM Tris HCl, 0.01% Tween 80, pH 7.4 at 25° C. in a total volume of 300 μl. Amidolytic activity is measured as the change in absorbance at 405 nm.

The apparent inhibition constant, Kᵢ′, are determined using the non-linear regression data analysis program Enzfitter® (Biosoft, Cambridge, UK). Kᵢ values are obtained by correcting for the effect of substrate according to the equation:

Kᵢ=Kᵢ′/(1+[S]/Kₘ)

SDS-Analysis

Samples are diluted with sample buffer and applied on a 4-12% SDS gel in MES buffer. The gel is coomassie stained afterwards. One of the proteins in the MW standard is aprotinin.

HPLC-analysis

Purity is checked on an analytical column C4 Jupiter 5 μm 4.6×250 mm. Solvent system is TFA/CH3CN, flow 1.5 ml/min. Gradient 5% B-55% B over 20 min. The purity measured by HPLC reflects the SDS pattern.

Table 2 shows the templates and primers used in the PCR's for construction of the plasmid pMaUJ72 and the yeast plasmid pMaUJ238.

Table 3 shows the DNA sequence of the primers used for construction and sequencing of the plasmids pMaUJ72, pMaUJ238, pMaUJ365 and pMaUJ367.

Table 4 shows yeast transformants harboring pMaUJ238, pMaUJ365 and pMaUJ367, respectively.

TABLE 2

| Final | PCR | | Upstream | Downstream |
|---|---|---|---|---|
| construct | reaction | Template | primer | primer |
| pMaUJ72 | | testis cDNA | oMaUJ41 | oMaUJ42 |
| pMaUJ238 | 1 | pEA314 | oMaUJ87 | oMaUJ88 |
| (212L-HKI18) | 2 | pMaUJ72 | oMaUJ89 | oMaUJ90 |
| | SOE | PCR product 1 + 2 | oMaUJ87 | oMaUJ90 |

TABLE 3

| | |
|---|---|
| Primer | S qu nc |

| | |
|---|---|
| oMaUJ11 | 5′ ACGCCAAGCTTTGGAGCC 3′ |
| oMaUJ41 | 5′ CCTTGATAGGCCCAGCCGGCCTACCCCGTGCGGTGCCTGC 3′ |
| oMaUJ42 | 5′ GGATGTCAAGCGGCCGCAGATCCCTGGCAG-CTGCTCATG 3′ |
| oMaUJ87 | 5′ CTGCAGAAGCACCATCAGGTTGGTG 3′ |
| oMaUJ88 | 5′ TCTCTTCTCCAATCTCTCAGCCATGGC 3′ |
| oMaUJ89 | 5′ CATGGCTGAGAGATTGGAGAAGAGATACCCCGTGCGG-TGCCTGCTGC 3′ |
| oMaUJ90 | 5′ CAGGCTGATCTAGACTTAAGATCCCTGGCAGCTGCTCA-TGCAC 3′ |
| oMaUJ125 | 5′ CAGGAATTCCATTCAAGAATAGTTC 3′ |
| oMaUJ126 | 5′ CCGTAGTCATCAATTTATTTTACATAACAC 3′ |
| oMaUJ224 | 5′ CTTTGGCTAACGTCGCCATGGCTGAGAGATTGGAGAAGAGATAC 3′ |
| oMaUJ225 | 5′ GGGCAGCAGGCACCGCACGGGGTATCTCTTCTCCAATCTCTC 3′ |
| oMaUJ226 | 5′ CCGTGCGGTGCCTGCTGCCCCCTGCCACTGGCTCTTGCAAAG 3′ |
| oMaUJ227 | 5′ GAAGTACCAGCGGGCAGCCCAGGCTTTGCAAGAGCCAGTGGCAG 3′ |
| oMaUJ228 | 5′ GGGCTGCCCGCTGGTACTTCGTTGCCTCTGTGGGCCAATGTAAC 3′ |
| oMaUJ229 | 5′ CATGACAGCCGCCATACCAGAAGCGGTTACATTGGCCCACAGAGG 3′ |
| oMaUJ230 | 5′ CTGGTATGGCGGCTGTCATGGCAATGCCAATAACTTTGCCTCGGAG 3′ |
| oMaUJ231 | 5′ CAGCTGCTCATGCACTCTTGCTCCGAGGCAAAGTTATTGG 3′ |
| oMaUJ232 | 5′ CAAGAGTGCATGAGCAGCTGCCAGGGATCTTAAGTCTAGA 3′ |
| oMaUJ233 | 5′ CACGGTCTTAGTTTCTAGACTTAAGATCCCTGG 3′ |
| oMaUJ234 | 5′ CCGTGCGGTGCCTGCTGCCCCCTGCCACTGGCCCTTGCAAAG 3′ |
| oMaUJ235 | 5′ GAAGTACCAGCGGGCAGCCCTGGCTTTGCAAGGGCCAGTGGCAG 3′ |
| oMaUJ236 | 5′ CATGACAGCCGCCATACACGAAGCGGTTACATTGGCCCACAGAGG 3′ |
| oMaUJ237 | 5′ CGTGTATGGCGGCTGTCATGGCAATGCCAATAACTTTGCCTCGGAG 3′ |

TABLE 4

| YEAST STRAIN | PLASMID | HKI18 ALLELE |
|---|---|---|
| yMaUJ33 | pMaUJ238 | HKI18 |
| yMaUJ69 | pMaUJ365 | HKI18-1 |
| yMaUJ70 | pMaUJ367 | HKI18-2 |

### EXEMPLARY ASPECTS OF THE INVENTION

The following is a nonlimiting list of some of the exemplary aspects and features of the invention:

1. An isolated polypeptide comprising the amino acid sequence of SEQ ID NO:2 or a variant thereof, wherein said sequence is at least 80% identical to residues 5 through 55 of SEQ ID NO:1.

2. The isolated polypeptide according to claim 1, wherein said polypeptide comprises a kunitz domain.

3. The isolated polypeptide according to any one of the claims 1-2, wherein said polypeptide has proteinase inhibiting activity.

4. The isolated polypeptide according to claim 3, which inhibits at least one of the proteases selected from the group consisting of chymotrypsin, elastase, cathepsin G, proteinase 3, plasmin, plasma kallikrein, glandular kallikrein and trypsin.

5. The isolated polypeptide according to any one of the claims 1-4, wherein said polypeptide is from 51 to 81 amino acid residues in length.

6. The isolated polypeptide according to any one of the claims 1-5, wherein said polypeptide is from 51 to 67 residues in length.

7. The isolated polypeptide according to any one of the claims 1-6, wherein Xaa5 of SEQ ID NO:2 is Pro.

8. The isolated polypeptide according to any one of the claims 1-7, wherein Xaa7 of SEQ ID NO:2 is Thr.

9. The isolated polypeptide according to any one of the claims 1-8, wherein Xaa9 of SEQ ID NO:2 is Pro.

10. The isolated polypeptide according to any one of the claims 1-9, wherein Xaa11 of SEQ ID NO:2 is Arg.

11. The isolated polypeptide according to any one of the claims 1-10, wherein Xaa11 of SEQ ID NO:2 is Lys.

12. The isolated polypeptide according to any one of the claims 1-11, wherein Xaa12 of SEQ ID NO:2 is Ala.

13. The isolated polypeptide according to any one of the claims 1-12, wherein Xaa13 of SEQ ID NO:2 Arg.

14. The isolated polypeptide according to any one of the claims 1-13, wherein Xaa14 of SEQ ID NO:2 is Ile.

15. The isolated polypeptide according to any one of the claims 1-14, wherein Xaa15 of SEQ ID NO:2 is Ile.

16. The isolated polypeptide according to any one of the claims 1-15, wherein Xaa30 of SEQ ID NO:2 is Val.

17. The isolated polypeptide according to any one of the claims 1-16, wherein Xaa35 of SEQ ID NO:2 is Arg.

18. The isolated polypeptide according to any one of the claims 1-6, wherein said sequence comprises residues 5 through 55 of SEQ ID NO:1.

19. The isolated polypeptide according to any one of the claims 1-6, wherein said sequence comprises residues 1 through 58 of SEQ ID NO:1.

20. The isolated polypeptide according to any one of the claims 1-6, wherein said sequence comprises a sequence independently selected from SEQ ID NO:4, SEQ ID NO:5, SEQ ID NO:6, or SEQ ID NO:7.

21. An isolated polypeptide obtainable by cultivation of a host cell comprising a polynucleotide construct encoding a polypeptide according to any one of the claims 1-20 in an appropriate growth medium under conditions allowing expression of said polynucleotide construct and recovering said polypeptide from the culture medium.

22. The isolated polypeptide according to claim 21, wherein said host cell is a eukaryotic cell.

23. The isolated polypeptide according to claim 21-22, wherein said host cell is a mammalian cell.

24. The isolated polypeptide according to claim 21-22, wherein said host cell is a yeast cell.

25. The isolated polypeptide according to claim 24, wherein said host cell is a strain of Saccharomyces cerevisiae.

26. A polynucleotide construct encoding a polypeptide according to any one of the claims 1-20.

27. The polynucleotide construct according to claim 26, which is a vector.

28. A host cell comprising the polynucleotide construct according to any one of the claims 26-27.

29 The host cell according to claim 28, which is a eukaryotic cell.

30. The host cell according to claim 29, which is of mammalian origin.

31. The host cell according to claim 29, which is a yeast cell.

32. The host cell according to claim 31, which is a strain of Saccharomyces cerevisiae.

33. A method for producing the isolated polypeptide according to any one of claims 1-25, the method comprising cultivating a host cell as defined in any one of claims 28-32 in an appropriate growth medium under conditions allowing expression of said polynucleotide construct and recovering said polypeptide from the culture medium.

34. A composition comprising an isolated polypeptide comprising the amino acid sequence of SEQ ID NO:2 or a variant thereof, wherein said sequence is at least 80% identical to residues 5 through 55 of SEQ ID NO:1.

35. A composition comprising an isolated polypeptide having the amino acid sequence of SEQ ID NO:2 or a variant thereof, wherein said sequence is at least 80% identical to residues 5 through 55 of SEQ ID NO:1.

36. A composition comprising an isolated polypeptide according to any one of the claims 1-25.

37. A pharmaceutical composition comprising an isolated polypeptide comprising the amino acid sequence of SEQ ID NO:2 or a variant thereof, wherein said sequence is at least 80% identical to residues 5 through 55 of SEQ ID NO:1; and optionally, a pharmaceutically acceptable carrier or vehicle.

38. A pharmaceutical composition comprising an isolated polypeptide according to any one of the claims 1-25; and optionally, a pharmaceutically acceptable carrier or vehicle.

39. Use of an isolated polypeptide comprising the amino acid sequence of SEQ ID NO:2 or a variant thereof, wherein said sequence is at least 80% identical to residues 5 through 55 of SEQ ID NO:1 for the preparation of a medicament for the treatment of systemic inflammatory response syndrome, acute pancreatitis, shock syndrome, disseminated intravascular coagulation, hyperfibrinolytic hemorrhage, myocardial infarction or for prevention of blood loss during major surgery.

40. Use of an isolated polypeptide as defined in any one of the claims 1-25 for the preparation of a medicament for the treatment of systemic inflammatory response syndrome, acute pancreatitis, shock syndrome, hyperfibrinolytic hemorrhage, myocardial infarction or for prevention of blood loss during major surgery.

41. A method for the treatment of systemic inflammatory response syndrome, acute pancreatitis, shock syndrome, hyperfibrinolytic hemorrhage, myocardial infarction or for prevention of blood loss during major surgery, the method comprising administering a therapeutically or prophylactically effective amount of an isolated polypeptide comprising the amino acid sequence of SEQ ID NO:2 or a variant thereof, wherein said sequence is at least 80% identical to residues 5 through 55 of SEQ ID NO:1; to a subject in need thereof.

42. A method for the treatment of systemic inflammatory response syndrome, acute pancreatitis, shock syndrome, hyperfibrinolytic hemorrhage, myocardial infarction or for prevention of blood loss during major surgery, the method comprising administering a therapeutically or prophylactically effective amount of an isolated polypeptide as defined in any one of claims 1-25 to a subject in need thereof.

43. A method of promoting the sale and/or use of a compound according to any of the preceding aspects, or otherwise described herein, comprising distributing information related to the use of the compound in the prevention or treatment of any condition or combination of conditions recited in any of the foregoing aspects or described elsewhere herein.

44. A pharmaceutical product comprising (a) a composition according to any of the foregoing aspects or elsewhere described herein, (b) a pharmaceutically acceptable carrier, vehicle, excipient, diluent, preservant, stabilizer, or combination of any thereof (including any multiples thereof—e.g., two diluents), and, optionally, (c) a notice associated with said container in form prescribed by a governmental agency regulating the manufacture, use, or sale of pharmaceuticals, which notice is reflective of approval by said agency of said pharmaceutical product for human or veterinary administration to treat at least one condition recited in any of the foregoing aspects or other condition or disease described herein.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Unless explicitly indicated or clearly contradicted by context, approximate values described herein (e.g., “about 10”) can be replaced by corresponding exact values and visa versa.

All methods described herein can be performed in any suitable order unless otherwise indicated herein or otherwise clearly contradicted by context. Any combination of the above-described elements in all possible variations thereof is encompassed by the invention unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All amino acid or nucleotide sequences of one of the aforementioned sequence patterns are to be considered individually disclosed herein. Thus, for example, an amino acid sequence pattern of three residues, where a “Xaa” represents one of the amino acid positions in the pattern represents a disclosure of at least twenty different sequences (i.e., one sequence for each naturally occurring amino acid residue that could be present in the Xaa position).

Preferred aspects and features of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law.

What is claimed is:

1. An isolated polypeptide comprising at least one Kunitz domain that comprises an amino acid sequence according to the formula Cys Xaa2 Xaa3 Xaa4 Xaa5 Xaa6 Xaa7 Xaa8 Xaa9 Cys Xaa11 Xaa12 Xaa13 Xaa14 Xaa15 Xaa16 Xaa17 Xaa18 Xaa19 Xaa20 Xaa21 Xaa22 Xaa23 Xaa24 Xaa25 Cys Xaa27 Xaa28 Phe Xaa30 Xaa31 Xaa32 Gly Cys Xaa35 Xaa36 Xaa37 Xaa38 Asn Xaa40 Xaa41 Xaa42 Xaa43 Xaa44 Xaa45 Xaa46 Cys Xaa48 Xaa49 Xaa50 Cys (SEQ ID NO:2), wherein the amino acid sequence is at least about 80% identical to the sequence of residues 5-55 of SEQ ID NO:1.

2. The polypeptide of claim 1, wherein the amino acid sequence is characterized by one or more of the following conditions: Xaa2 is an Ala, Val, Leu, Ser, Thr, Asn, Lys, Glu, Gln, Arg, Phe, Tyr, or Met residue, or is absent; Xaa3 is an Ala, Val, Leu, Ser, Thr, Asp, Glu, Gln Phe, or Met residue, or is absent; Xaa4 is a Gly, Ala, Leu, Ser, Asp, Lys, Glu, Gln or Pro residue, or is absent; Xaa5 is Ala, Val, Leu, Glu, Ser, Asn, Lys, Glu, Tyr, Met, Pro, or is absent; Xaa6 is an Ala, Val, Leu, Ser, Asp, Asn, Lys, Glu, Arg, Tyr, or Met residue, or is absent; Xaa7 is an Ala, Val, Thr, Asp, Lys, Glu, Gln, Arg, His, Tyr, or Pro residue, or is absent; Xaa8 is a Gly or Asp residue, or is absent; Xaa9 is a Leu, Glu, Ser, Thr, Asn, Gln, Arg, or Pro residue, or is absent; Xaa11 is a Gly, Ala, Leu, Ser, Thr, Asn, Lys, Glu, Gln, Arg, or Met residue, or is absent; Xaa12 is a Gly, Ala, Thr, Asp, Glu, or His residue, or is absent; Xaa13 is a Leu, Glu, Ser, Asn, Glu, Arg, Phe, Trp, Tyr, or Met residue, or is absent; Xaa14 is an Ala, Val, Leu, Glu, Thr, Glu, Phe, or Met residue, or is absent; Xaa15 is an Ala, Val, Leu, Glu, Ser, Thr, Asn, Lys, Glu, Gln or Pro residue, or is absent; Xaa16 is a Leu, Lys, Arg, or His residue, or is absent; Xaa17 is a Phe, Trp, or Tyr residue, or is absent; Xaa18 is an Ala, His, Phe, Trp, or Tyr residue, or is absent; Xaa19 is a Phe or Tyr residue, or is absent; Xaa20 is a Val, Ser, Asp, Asn, or Arg residue, or is absent; Xaa21 is a Gly, Ala, Leu, Glu, Ser, Asn, Lys, Phe, or Pro residue, or is absent; Xaa22 is a Val, Leu, Ser, Thr, Asn, Lys, Glu, Gln, Arg, Phe, or Tyr residue, or is absent; Xaa23 is an Ala, Val, Leu, Glu, Ser, Thr, Asp, Asn, Lys, Glu, Arg, or Tyr residue, or is absent; Xaa24 is a Gly, Asn, Lys, Glu, Gln, Arg, Tyr, or Met residue, or is absent; Xaa25 is an Ala, Leu, Glu, Ser, Thr, Lys, Glu, Gln, Arg, or His residue, or is absent; Xaa27 is an Ala, Val, Ser, Thr, Asp, Asn, Lys, Glu, Gln, Arg, or His residue, or is absent; Xaa28 is an Ala, Leu, Ser, Thr, Asn, Lys, Glu, Gln, Arg, Met, or Pro residue, or is absent; Xaa30 is an Ala, Val, Leu, Glu, Thr, Lys, Gln, Phe, Trp, or Pro residue, or is absent; Xaa31 is a Ser, Phe, or Tyr residue, or is absent; Xaa32 is a Gly, Ser, Thr, or Arg residue, or is absent; Xaa35 is a Gly, Leu, Asp, Asn, Glu, Gln, Arg, His, Tyr, or Met residue, or is absent; Xaa36 is a Gly, Ala, or Arg residue, or is absent; Xaa37 is a Ser, Asp, Asn, or Lys residue, or is absent; Xaa38 is a Gly, Ala, Ser, Asp, Asn, Lys, Glu, Gln or Arg residue, or is absent; Xaa40 is a Ser, Asn, Lys, or Arg residue, or is absent; Xaa41 is a Phe or Tyr residue, or is absent; Xaa42 is a Gly, Ala, Val, Leu, Thr, Asp, Asn, Lys, Glu, Gln, Arg, His, Tyr, or Pro residue, or is absent; Xaa43 is a Ser, Thr, Asp, Asn, Glu, or Arg residue, or is absent; Xaa44 is an Ala, Leu, Lys, Glu, Gln, Arg, or Trp residue, or is absent; Xaa45 is an Ala, Asp, Lys, Glu, or Gln residue, or is absent; Xaa46 is an Ala, Ser, Thr, Asp, Asn, Lys, Glu, Gln or Tyr residue, or is absent; Xaa48 is a Leu, Ile, Glu, Asp, Lys, Glu, Gln, Arg, or Met residue, or is absent; Xaa49 is a Gly, Ala, Leu, Ser, Thr, Asp, Asn, Lys, Glu, Gln or Arg residue, or is absent; and Xaa50 is an Ala, Ser, Thr, Val, Glu, Lys, Arg, Phe, or Met residue, or is absent.

3. The isolated polypeptide according to claim 2, wherein Xaa2 is an Ala, Val, Leu, Ser, Thr, Asn, Lys, Glu, Gln, Arg, Phe, Tyr, or Met residue, or is absent; Xaa3 is an Ala, Val, Leu, Ser, Thr, Asp, Glu, Gln Phe, or Met residue, or is absent; Xaa4 is a Gly, Ala, Leu, Ser, Asp, Lys, Glu, Gln or Pro residue, or is absent; Xaa5 is Ala, Val, Leu, Glu, Ser, Asn, Lys, Glu, Tyr, Met, Pro, or is absent; Xaa6 is an Ala, Val, Leu, Ser, Asp, Asn, Lys, Glu, Arg, Tyr, or Met residue, or is absent; Xaa7 is an Ala, Val, Thr, Asp, Lys, Glu, Gln, Arg, His, Tyr, or Pro residue, or is absent; Xaa8 is a Gly or Asp residue, or is absent; Xaa9 is a Leu, Glu, Ser, Thr, Asn, Gln, Arg, or Pro residue, or is absent; Xaa11 is a Gly, Ala, Leu, Ser, Thr, Asn, Lys, Glu, Gln, Arg, or Met residue, or is absent; Xaa2 is a Gly, Ala, Thr, Asp, Glu, or His residue, or is absent; Xaa13 is a Leu, Glu, Ser, Asn, Glu, Arg, Phe, Trp, Tyr, or Met residue, or is absent; Xaa14 is an Ala, Val, Leu, Glu, Thr, Glu, Phe, or Met residue, or is absent; Xaa15 is an Ala, Val, Leu, Glu, Ser, Thr, Asn, Lys, Glu, Gln or Pro residue, or is absent; Xaa16 is a Leu, Lys, Arg, or His residue, or is absent; Xaa17 is a Phe, Trp, or Tyr residue, or is absent; Xaa18 is an Ala, His, Phe, Trp, or Tyr residue, or is absent; Xaa19 is a Phe or Tyr residue, or is absent; Xaa20 is a Val, Ser, Asp, Asn, or Arg residue, or is absent; Xaa21 is a Gly, Ala, Leu, Glu, Ser, Asn, Lys, Phe, or Pro residue, or is absent; Xaa22 is a Val, Leu, Ser, Thr, Asn, Lys, Glu, Gln, Arg, Phe, or Tyr residue, or is absent; Xaa23 is an Ala, Val, Leu, Glu, Ser, Thr, Asp, Asn, Lys, Glu, Arg, or Tyr residue, or is absent; Xaa24 is a Gly, Asn, Lys, Glu, Gln Arg, Tyr, or Met residue, or is absent; Xaa25 is an Ala, Leu, Glu, Ser, Thr, Lys, Glu, Gln Arg, or His residue, or is absent; Xaa27 is an Ala, Val, Ser, Thr, Asp, Asn, Lys, Glu, Gln, Arg, or His residue, or is absent; Xaa28 is an Ala, Leu, Ser, Thr, Asn, Lys, Glu, Gln, Arg, Met, or Pro residue, or is absent; Xaa30 is an Ala, Val, Leu, Glu, Thr, Lys, Gln Phe, Trp, or Pro residue, or is absent; Xaa31 is a Ser, Phe, or Tyr residue, or is absent; Xaa32 is a Gly, Ser, Thr, or Arg residue, or is absent; Xaa35 is a Gly, Leu, Asp, Asn, Glu, Gln, Arg, His, Tyr, or Met residue, or is absent; Xaa36 is a Gly, Ala, or Arg residue, or is absent; Xaa37 is a Ser, Asp, Asn, or Lys residue, or is absent; Xaa38 is a Gly, Ala, Ser, Asp, Asn, Lys, Glu, Gln or Arg residue, or is absent; Xaa40 is a Ser, Asn, Lys, or Arg residue, or is absent; Xaa41 is a Phe or Tyr residue, or is absent; Xaa42 is a Gly, Ala, Val, Leu, Thr, Asp, Asn, Lys, Glu, Gln Arg, His, Tyr, or Pro residue, or is absent; Xaa43 is a Ser, Thr, Asp, Asn, Glu, or Arg residue, or is absent; Xaa44 is an Ala, Leu, Lys, Glu, Gln, Arg, or Trp residue, or is absent; Xaa45 is an Ala, Asp, Lys, Glu, or Gln residue, or is absent; Xaa46 is an Ala, Ser, Thr, Asp, Asn, Lys, Glu, Gln or Tyr residue, or is absent; Xaa48 is a Leu, Ile, Glu, Asp, Lys, Glu, Gln, Arg, or Met residue, or is absent; Xaa49 is a Gly, Ala, Leu, Ser, Thr, Asp, Asn, Lys, Glu, Gln or Arg residue, or is absent; and Xaa50 is an Ala, Ser, Thr, Val, Glu, Lys, Arg, Phe, or Met residue, or is absent.

4. The isolated polypeptide of claim 3, wherein the polypeptide detectably inhibits the activity of at least one of the proteases selected from the group consisting of chymotrypsin, elastase, cathepsin G, proteinase 3, plasmin, plasma kallikrein, glandular kallikrein and trypsin.

5. The isolated polypeptide of claim 3, wherein the polypeptide is from 51 to about 80 amino acid residues in length.

6. The isolated polypeptide of claim 5, wherein the polypeptide is from 51 to about 70 residues in length.

7. The isolated polypeptide of claim 3, wherein the amino acid sequence is characterized by one or more of the following Xaa5 is Pro; Xaa7 is Thr; Xaa9 is Pro; Xaa11 is Arg or Lys; Xaa12 of SEQ ID NO:2 is Ala; Xaa13 of SEQ ID NO:2 is Arg; Xaa14 of SEQ ID NO:2 is Ile; Xaa15 of SEQ ID NO:2 is Ile; Xaa30 of SEQ ID NO:2 is Val; and Xaa35 of SEQ ID NO:2 is Arg.

8. The isolated polypeptide of claim 7, wherein Xaa5 is Pro; Xaa7 is Thr; Xaa9 is Pro; Xaa11 is Arg or Lys; Xaa12 of SEQ ID NO:2 is Ala; Xaa13 of SEQ ID NO:2 is Arg;
Xaa14 of SEQ ID NO:2 is Ile; Xaa15 of SEQ ID NO:2 is Ile; Xaa30 of SEQ ID NO:2 is Val; and Xaa35 of SEQ ID NO:2 is Arg.

9. The isolated polypeptide of claim 1, wherein the amino acid sequence comprises residues 5-55 of SEQ ID NO:1.

10. The isolated polypeptide of claim 9, wherein the amino acid sequence comprises residues 1-58 of SEQ ID NO:1.

11. The isolated polypeptide of claim 1, wherein the amino acid sequence comprises SEQ ID NO:4, SEQ ID NO:5, SEQ ID NO:6, or SEQ ID NO:7.

12. A nucleic acid encoding the polypeptide of claim 3.

13. A vector comprising the nucleic acid of claim 12.

14. A host cell comprising the nucleic acid of claim 12.

15. A composition comprising the polypeptide of claim 1 and a pharmaceutically acceptable carrier, vehicle, diluent, excipient, or a combination of any thereof.

16. A composition comprising the polypeptide of claim 3 and a pharmaceutically acceptable carrier, vehicle, diluent, excipient, or a combination of any thereof.

17. A composition comprising the polypeptide of claim 9 and a pharmaceutically acceptable carrier, vehicle, diluent, excipient, or a combination of any thereof.

18. A method for inducing, promoting, and/or enhancing at least one physiological response associated with the treatment or prevention of systemic inflammatory response syndrome, acute pancreatitis, shock syndrome, hyperfibrinolytic hemorrhage, or myocardial infarction or for preventing blood loss in a subject comprising administering the composition of claim 15 to the subject in an amount sufficient to induce, promote, and/or enhance the physiological response.

19. A method for inducing, promoting, and/or enhancing at least one physiological response associated with the treatment or prevention of systemic inflammatory response syndrome, acute pancreatitis, shock syndrome, hyperfibrinolytic hemorrhage, or myocardial infarction or for preventing blood loss in a subject comprising administering the composition of claim 16 to the subject in an amount sufficient to induce, promote, and/or enhance the physiological response.

20. A method for inducing, promoting, and/or enhancing at least one physiological response associated with the treatment or prevention of systemic inflammatory response syndrome, acute pancreatitis, shock syndrome, hyperfibrinolytic hemorrhage, or myocardial infarction or for preventing blood loss in a subject comprising administering the composition of claim 17 to the subject in an amount sufficient to induce, promote, and/or enhance the physiological response.
